SEQUENCE LISTING (82 sequences)

SEQ ID NO: 1 - protein, 11 aa, Artificial Sequence
  Peptide linker
SGGGGTGGGGS

SEQ ID NO: 2 - protein, 11 aa, Artificial Sequence
  Peptide linker
SGGGGSGGGGS

SEQ ID NO: 3 - protein, 8 aa, Artificial Sequence
  Hinge linker
DKTHTCAA

SEQ ID NO: 4 - protein, 11 aa, Artificial Sequence
  Hinge linker
DKTHTCPPCPA

SEQ ID NO: 5 - protein, 18 aa, Artificial Sequence
  Hinge linker
DKTHTCPPCPATCPPCPA

SEQ ID NO: 6 - protein, 25 aa, Artificial Sequence
  Hinge linker
DKTHTCPPCPATCPPCPATCPPCPA

SEQ ID NO: 7 - protein, 30 aa, Artificial Sequence
  Hinge linker
DKTHTCPPCPAGKPTLYNSLVMSDTAGTCY

SEQ ID NO: 8 - protein, 31 aa, Artificial Sequence
  Hinge linker
DKTHTCPPCPAGKPTHVNVSVVMAEVDGTCY

SEQ ID NO: 9 - protein, 15 aa, Artificial Sequence
  Hinge linker
DKTHTCCVECPPCPA

SEQ ID NO: 10 - protein, 26 aa, Artificial Sequence
  Hinge linker
DKTHTCPRCPEPKSCDTPPPCPRCPA

SEQ ID NO: 11 - protein, 11 aa, Artificial Sequence
  Hinge linker
DKTHTCPSCPA

SEQ ID NO: 12 - protein, 7 aa, Artificial Sequence
  Flexible linker
SGGGGSE

SEQ ID NO: 13 - protein, 6 aa, Artificial Sequence
  Flexible linker
DKTHTS

SEQ ID NO: 14 - protein, 6 aa, Artificial Sequence
  Flexible linker
SGGGGS

SEQ ID NO: 15 - protein, 11 aa, Artificial Sequence
  Flexible linker
SGGGGSGGGGS

SEQ ID NO: 16 - protein, 16 aa, Artificial Sequence
  Flexible linker
SGGGGSGGGGSGGGGS

SEQ ID NO: 17 - protein, 21 aa, Artificial Sequence
  Flexible linker
SGGGGSGGGGSGGGGSGGGGS

SEQ ID NO: 18 - protein, 26 aa, Artificial Sequence
  Flexible linker
SGGGGSGGGGSGGGGSGGGGSGGGGS

SEQ ID NO: 19 - protein, 11 aa, Artificial Sequence
  Flexible linker
AAAGSGGASAS

SEQ ID NO: 20 - protein, 16 aa, Artificial Sequence
  Flexible linker
  misc_feature (7)..(7): Xaa can be any naturally occurring amino acid
AAAGSGXGGGSGASAS

SEQ ID NO: 21 - protein, 21 aa, Artificial Sequence
  Flexible linker
  misc_feature (7)..(7): Xaa can be any naturally occurring amino acid
  misc_feature (12)..(12): Xaa can be any naturally occurring amino acid
AAAGSGXGGGSXGGGSGASAS

SEQ ID NO: 22 - protein, 26 aa, Artificial Sequence
  Flexible linker
  misc_feature (7)..(7): Xaa can be any naturally occurring amino acid
  misc_feature (12)..(12): Xaa can be any naturally occurring amino acid
  misc_feature (17)..(17): Xaa can be any naturally occurring amino acid
AAAGSGXGGGSXGGGSXGGGSGASAS

SEQ ID NO: 23 - protein, 31 aa, Artificial Sequence
  Flexible linker
  misc_feature (7)..(7): Xaa can be any naturally occurring amino acid
  misc_feature (12)..(12): Xaa can be any naturally occurring amino acid
  misc_feature (17)..(17): Xaa can be any naturally occurring amino acid
  misc_feature (22)..(22): Xaa can be any naturally occurring amino acid
AAAGSGXGGGSXGGGSXGGGSXGGGSGASAS

SEQ ID NO: 24 - protein, 13 aa, Artificial Sequence
  Flexible linker
  misc_feature (7)..(7): Xaa can be any naturally occurring amino acid
AAAGSGXSGASAS

SEQ ID NO: 25 - protein, 28 aa, Artificial Sequence
  Flexible linker
PGGNRGTTTTRRPATTTGSSPGPTQSHY

SEQ ID NO: 26 - protein, 11 aa, Artificial Sequence
  Flexible linker
ATTTGSSPGPT

SEQ ID NO: 27 - protein, 6 aa, Artificial Sequence
  Flexible linker
ATTTGS

SEQ ID NO: 28 - protein, 21 aa, Artificial Sequence
  Flexible linker
EPSGPISTINSPPSKESHKSP

SEQ ID NO: 29 - protein, 15 aa, Artificial Sequence
  Flexible linker
GTVAAPSVFIFPPSD

SEQ ID NO: 30 - protein, 15 aa, Artificial Sequence
  Flexible linker
GGGGIAPSMVGGGGS

SEQ ID NO: 31 - protein, 15 aa, Artificial Sequence
  Flexible linker
GGGGKVEGAGGGGGS

SEQ ID NO: 32 - protein, 15 aa, Artificial Sequence
  Flexible linker
GGGGSMKSHDGGGGS

SEQ ID NO: 33 - protein, 15 aa, Artificial Sequence
  Flexible linker
GGGGNLITIVGGGGS

SEQ ID NO: 34 - protein, 15 aa, Artificial Sequence
  Flexible linker
GGGGVVPSLPGGGGS

SEQ ID NO: 35 - protein, 12 aa, Artificial Sequence
  Flexible linker
GGEKSIPGGGGS

SEQ ID NO: 36 - protein, 18 aa, Artificial Sequence
  Flexible linker
RPLSYRPPFPFGFPSVRP

SEQ ID NO: 37 - protein, 18 aa, Artificial Sequence
  Flexible linker
YPRSIYIRRRHPSPSLTT

SEQ ID NO: 38 - protein, 18 aa, Artificial Sequence
  Flexible linker
TPSHLSHILPSFGLPTFN

SEQ ID NO: 39 - protein, 18 aa, Artificial Sequence
  Flexible linker
RPVSPFTFPRLSNSWLPA

SEQ ID NO: 40 - protein, 18 aa, Artificial Sequence
  Flexible linker
SPAAHFPRSIPRPGPIRT

SEQ ID NO: 41 - protein, 18 aa, Artificial Sequence
  Flexible linker
APGPSAPSHRSLPSRAFG

SEQ ID NO: 42 - protein, 18 aa, Artificial Sequence
  Flexible linker
PRNSIHFLHPLLVAPLGA

SEQ ID NO: 43 - protein, 18 aa, Artificial Sequence
  Flexible linker
MPSLSGVLQVRYLSPPDL

SEQ ID NO: 44 - protein, 18 aa, Artificial Sequence
  Flexible linker
SPQYPSPLTLTLPPHPSL

SEQ ID NO: 45 - protein, 18 aa, Artificial Sequence
  Flexible linker
NPSLNPPSYLHRAPSRIS

SEQ ID NO: 46 - protein, 17 aa, Artificial Sequence
  Flexible linker
LPWRTSLLPSLPLRRRP

SEQ ID NO: 47 - protein, 18 aa, Artificial Sequence
  Flexible linker
PPLFAKGPVGLLSRSFPP

SEQ ID NO: 48 - protein, 18 aa, Artificial Sequence
  Flexible linker
VPPAPVVSLRSAHARPPY

SEQ ID NO: 49 - protein, 17 aa, Artificial Sequence
  Flexible linker
LRPTPPRVRSYTCCPTP

SEQ ID NO: 50 - protein, 18 aa, Artificial Sequence
  Flexible linker
PNVAHVLPLLTVPWDNLR

SEQ ID NO: 51 - protein, 18 aa, Artificial Sequence
  Flexible linker
CNPLLPLCARSPAVRTFP

SEQ ID NO: 52 - protein, 11 aa, Artificial Sequence
  Rigid linker
GAPAPAAPAPA

SEQ ID NO: 53 - protein, 4 aa, Artificial Sequence
  Rigid linker
PPPP

SEQ ID NO: 54 - protein, 11 aa, Artificial Sequence
  Albumin binding peptide
DLCLRDWGCLW

SEQ ID NO: 55 - protein, 11 aa, Artificial Sequence
  Albumin binding peptide
DICLPRWGCLW

SEQ ID NO: 56 - protein, 15 aa, Artificial Sequence
  Albumin binding peptide
MEDICLPRWGCLWGD

SEQ ID NO: 57 - protein, 20 aa, Artificial Sequence
  Albumin binding peptide
QRLMEDICLPRWGCLWEDDE

SEQ ID NO: 58 - protein, 20 aa, Artificial Sequence
  Albumin binding peptide
QGLIGDICLPRWGCLWGRSV

SEQ ID NO: 59 - protein, 21 aa, Artificial Sequence
  Albumin binding peptide
QGLIGDICLPRWGCLWGRSVK

SEQ ID NO: 60 - protein, 15 aa, Artificial Sequence
  Albumin binding peptide
EDICLPRWGCLWEDD

SEQ ID NO: 61 - protein, 18 aa, Artificial Sequence
  Albumin binding peptide
RLMEDICLPRWGCLWEDD

SEQ ID NO: 62 - protein, 16 aa, Artificial Sequence
  Albumin binding peptide
MEDICLPRWGCLWEDD

SEQ ID NO: 63 - protein, 15 aa, Artificial Sequence
  Albumin binding peptide
MEDICLPRWGCLWED

SEQ ID NO: 64 - protein, 18 aa, Artificial Sequence
  Albumin binding peptide
RLMEDICLARWGCLWEDD

SEQ ID NO: 65 - protein, 20 aa, Artificial Sequence
  Albumin binding peptide
EVRSFCTRWPAEKSCKPLRG

SEQ ID NO: 66 - protein, 20 aa, Artificial Sequence
  Albumin binding peptide
RAPESFVCYWETICFERSEQ

SEQ ID NO: 67 - protein, 11 aa, Artificial Sequence
  Albumin binding peptide
EMCYFPGICWM

SEQ ID NO: 68 - protein, 20 aa, Artificial Sequence
  Linker
GGGGSGGGGSGGGGSGGGGS

SEQ ID NO: 69 - protein, 16 aa, Artificial Sequence
  Linker
SGGGGSGGGGSGGGGS

SEQ ID NO: 70 - protein, 16 aa, Artificial Sequence
  Linker
SGGGGSGGGGTGGGGS

SEQ ID NO: 71 - protein, 10 aa, Artificial Sequence
  Antibody 645 CDRH1
GIDLSNYAIN

SEQ ID NO: 72 - protein, 16 aa, Artificial Sequence
  Antibody 645 CDRH2
IIWASGTTFYATWAKG

SEQ ID NO: 73 - protein, 13 aa, Artificial Sequence
  Antibody 645 CDRH3
TVPGYSTAPYFDL

SEQ ID NO: 74 - protein, 12 aa, Artificial Sequence
  Antibody 645 CDRL1
QSSPSVWSNFLS

SEQ ID NO: 75 - protein, 7 aa, Artificial Sequence
  Antibody 645 CDRL2
EASKLTS

SEQ ID NO: 76 - protein, 11 aa, Artificial Sequence
  Antibody 645 CDRL3
GGGYSSISDTT

SEQ ID NO: 77 - protein, 121 aa, Artificial Sequence
  Antibody 645 heavy chain variable domain
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSS

SEQ ID NO: 78 - protein, 121 aa, Artificial Sequence
  Antibody 645 heavy chain variable domain, mutated
EVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSS

SEQ ID NO: 79 - protein, 110 aa, Artificial Sequence
  Antibody 645 light chain variable domain
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGGGTKVEIK

SEQ ID NO: 80 - protein, 110 aa, Artificial Sequence
  Antibody 645 light chain variable domain, mutated
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIK

SEQ ID NO: 81 - protein, 251 aa, Artificial Sequence
  645 scFv (VH-VL)
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSGGGGSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGGGTKVEIK

SEQ ID NO: 82 - protein, 251 aa, Artificial Sequence
  645 dsscFv
EVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSSGGGGSGGGGSGGGGSGGGGSDIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIK

(19) United States
(12) Patent Application Publication
(10) Pub. No.: US 2023/0242677 A1
(43) Pub. Date: Aug. 3, 2023

(54) MULTI-SPECIFIC ANTIBODIES

(71) Applicant: UCB BIOPHARMA SRL, Brussels (BE)

(72) Inventors: Emily Mary Cairistine Barry, Slough, Berkshire (GB); Emma Dave, Slough, Berkshire (GB); Sam Philip Heywood, Slough, Berkshire (GB)

(21) Appl. No.: 17/787,094

(22) PCT Filed: Dec. 18, 2020
(86) PCT No.: PCT/EP2020/087134

§ 371 Date: Jun. 17, 2022

(30) Foreign Application Priority Data
Dec. 20, 2019 (GB) 1919058.6

(51) Int. Cl. C07K 16/46; C12N 15/63; C07K 1/22

(52) U.S. Cl. CPC C07K 16/468; C07K 1/22; C12N 15/63; C07K 2317/31; C07K 2317/624

(57) ABSTRACT

Multi-specific Antibodies The present disclosure relates to a multi-specific antibody comprising or consisting of: a) a polypeptide chain of formula (I): VH-CH1-(CH2)-(CH3)-(X)-(V1); and b) a polypeptide chain of formula (II): (V3)-(Z) -VL-C_{L}-(Y)-(V2) wherein the polypeptide chain of formula (II) comprises at least one dsscFv, dsFv, scFv, VH or VHH, and wherein the polypeptide chain of formula (I) comprises a protein A binding domain and wherein the polypeptide chain of formula (II) does not bind protein A. The disclosure also provides polynucleotide sequences encoding said multi-specific antibody, vectors comprising the polynucleotides and host cells comprising said vectors and/or polynucleotide sequences. The disclosure also provides pharmaceutical formulations comprising same, for example for use in treatment. There is also provided a method of expressing a multi-specific antibody of the present disclosure from a host cell.

### CROSS REFERENCE TO RELATED APPLICATIONS

This application is a U.S. National Stage of International Application No. PCT/EP2020/087134, filed Dec. 18, 2020, which claims priority to Great Britain Application No. 1919058.6, filed Dec. 20, 2019, the entire contents of each of which are fully incorporated herein by reference.

### INCORPORATION BY REFERENCE OF MATERIAL SUBMITTED ELECTRONICALLY

A Sequence Listing, which is a part of the present disclosure, is submitted concurrently with the specification as a text file. The name of the text file containing the Sequence Listing is “58051_Seqlisting.txt.” The Sequence Listing was created on Jun. 8, 2022, and is 29,519 bytes in size. The subject matter of the Sequence Listing is incorporated by reference herein in its entirety.

### FIELD OF INVENTION

The present disclosure relates to multi-specific antibodies, formulations comprising the same, polynucleotide sequences encoding said antibodies, vectors comprising said polynucleotide sequences and host cells comprising said vectors and/or polynucleotide sequences. The disclosure also relates to the use of the multi-specific antibodies and formulations in therapy. The disclosure extends to a method of expressing the multi-specific antibodies, for example in a host cell, and also extends to a method of purifying the multi-specific antibodies, said method comprising a protein A purification step.

### BACKGROUND OF INVENTION

There are a number of approaches for generating multi-specific, notably bi-specific antibodies. Morrison et al (Coloma and Morrison 1997, Nat Biotechnol. 15, 159-163) describes the fusion of single chain variable fragments (scFv) to whole antibodies, e.g. IgG. Schoonjans et al., 2000, Journal of Immunology, 165, 7050-7057, describes the fusion of scFv to antibody Fab fragments. WO2015/197772 describes the fusion of disulphide stabilised scFv (dsscFv) to Fab fragments.

Standard approaches described in the prior art comprise the expression in a host cell of at least two polypeptides, each one coding for a heavy chain (HC) or a light chain (LC) of a whole antibody or antigen binding fragment thereof e.g. a Fab, to which an additional antigen binding fragment of an antibody can be fused to the N- and/or C- terminal position of the heavy chain and/or the light chain. When trying to recombinantly produce such multi-specific antibodies by expressing two (one light chain and one heavy chain to form an appended Fab) or four polypeptides (two light chains and two heavy chains to form an appended IgG), it usually requires expressing the light chain in excess over the heavy chain, in order to ensure the proper folding of the heavy chain upon assembly with its corresponding light chain. In particular, the CH1 (domain 1 of the heavy chain constant region) is prevented from folding on itself by BIP proteins, which can be displaced by a corresponding LC; therefore, the correct folding of the CH1/HC is dependent on the availability of its corresponding LC (Lee et al., 1999, Molecular Biology of the Cell, Vol. 10, 2209-2219).

The present inventors have observed that those methods of expressing multi-specific antibodies may result in the production of the light chain in excess over the heavy chain, which remains in the host cell harvest, and that the excess of light chain tends to form dimeric complexes (or “LC dimers”) which are present as a by-product of the production process with the desired multi-specific antibody, notably monomeric, and thus need to be purified away.

Importantly, the technical problem associated with the formation of dimers of light chains, when fused on N- and/or C-terminal to additional antigen binding fragment(s), has not been identified so far, and the commonly used analytical methods have not allowed the detection and quantification of those appended LC dimers amongst the heterogenous products of the production process. This may result in a significant bias when estimating the amount of the products using standard analytical methods.

Thus, there is a need to improve multi-specific antibodies and methods of production thereof, which allow the isolation and removal of the appended LC dimers easily and efficiently at the earliest steps of the production process, and thus improve the yield of the protein of interest for use in therapy, which is the multi-specific antibody, in particular in its monomeric form.

### SUMMARY OF THE INVENTION

The present inventors have re-engineered the multi-specific antibodies concerned to provide improved multi-specific antibodies with equivalent functionality and stability, whilst increasing the yield of “multi-specific antibody” material, notably monomeric, obtained after purification, notably after a one-step purification comprising a protein A affinity chromatography.

Thus, in one aspect, there is provided a multi-specific antibody comprising or consisting of: a polypeptide chain of formula (I):

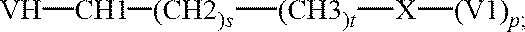

and a polypeptide chain of formula (II):

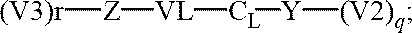

wherein:
VH represents a heavy chain variable domain;
CH1 represents domain 1 of a heavy chain constant region;
CH2 represents domain 2 of a heavy chain constant region;
CH3 represents domain 3 of a heavy chain constant region;
X represents a bond or linker;
V1 represents a dsscFv, a dsFv, a scFv, a VH, a VL or a VHH;
V3 represents a dsscFv, a dsFv, a scFv, a VH, a VL or a VHH;
Z represents a bond or linker;
VL represents a light chain variable domain;
C_{L} represents a domain from a light chain constant region, such as Ckappa;
Y represents a bond or linker;
V2 represents a dsscFv, a dsFv, a scFv, a VH, a VL or a VHH;
p represents 0 or 1;
q represents 0 or 1;
r represents 0 or 1;
s represents 0 or 1;
t represents 0 or 1;
wherein when p is 0, X is absent and when q is 0, Y is absent and when r is 0, Z is absent; and
wherein when q is 0, r is 1 and when r is 0, q is 1; and
wherein the polypeptide chain of formula (II) comprises at least one dsscFv, dsFv, scFv, VH, or VHH; and
wherein the polypeptide chain of formula (I) comprises a protein A binding domain; and
wherein the polypeptide chain of formula (II) does not bind protein A.

Advantageously, the multi-specific antibodies of the present disclosure can be more efficiently purified with a purification method which is improved over the methods commonly used in the prior art, notably in that the improved method comprises less steps, which is cost and time effective at the industrial scale. In particular, the multi-specific antibodies of the present disclosure maximise the quantity of proteins of interest (i-e, the correct multi-specific antibody format) obtained after a one-step purification method comprising a protein A affinity chromatography, whereby the purification of the multi-specific antibodies of interest and the removal of the appended LC dimers occur concurrently. Advantageously, the methods of production and purification of the multi-specific antibodies of the present disclosure do not require an additional purification step to capture the free, unbound light chains in excess, notably the appended LC dimers.

### DETAILED DESCRIPTION OF THE INVENTION

Antibodies for use in the context of the present disclosure include whole antibodies and functionally active fragments thereof (i.e., molecules that contain an antigen binding domain that specifically binds an antigen, also termed antigen-binding fragments). Features described herein with respect to antibodies also apply to antibody fragments unless context dictates otherwise. The antibody may be (or derived from), monoclonal, multi-valent, multi-specific, bispecific, fully human, humanized or chimeric.

Whole antibodies, also known as “immunoglobulins (Ig)” generally relate to intact or full-length antibodies i.e. comprising the elements of two heavy chains and two light chains, interconnected by disulphide bonds, which assemble to define a characteristic Y-shaped three-dimensional structure. Classical natural whole antibodies are monospecific in that they bind one antigen type, and bivalent in that they have two independent antigen binding domains. The terms “intact antibody”, “full-length antibody” and “whole antibody” are used interchangeably to refer to a monospecific bivalent antibody having a structure similar to a native antibody structure, including an Fc region as defined herein.

Each light chain is comprised of a light chain variable region (abbreviated herein as VL) and a light chain constant region (C_{L}). Each heavy chain is comprised of a heavy variable region (abbreviated herein as VH) and a heavy chain constant region (CH) constituted of three constant domains CH1, CH2 and CH3, or four constant domains CH1, CH2, CH3 and C_{H4}, depending on the Ig class. The “class” of an Ig or antibody refers to the type of constant region and includes IgA, IgD, IgE, IgG and IgM and several of them can be further divided into subclasses, e.g. IgG1, IgG2, IgG3, IgG4. The constant regions of the antibodies may mediate the binding of the immunoglobulin to host tissues or factors, including various cells of the immune system (e.g., effector cells) and the first component (Clq) of the classical complement system.

The VH and VL regions of the antibody or antigen-binding fragment thereof according to the present invention can be further subdivided into regions of hypervariability (or “hypervariable regions”) determining the recognition of the antigen, termed complementarity determining regions (CDR), interspersed with regions that are more structurally conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The CDRs and the FR together form a variable region. By convention, the CDRs in the heavy chain variable region of an antibody or antigen-binding fragment thereof are referred as CDR-H1, CDR-H2 and CDR-H3 and in the light chain variable region as CDR-L1, CDR-L2 and CDR-L3. They are numbered sequentially in the direction from the N-terminus to the C-terminus of each chain.

CDRs are conventionally numbered according to a system devised by Kabat et al. This system is set forth in Kabat et al., 1991, in Sequences of Proteins of Immunological Interest, US Department of Health and Human Services, NIH, USA (hereafter “Kabat et al. (supra)″). This numbering system is used in the present specification except where otherwise indicated. The Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence.

The CDRs of the heavy chain variable domain are located at residues 31-35 (CDR-H1), residues 50-65 (CDR-H2) and residues 95-102 (CDR-H3) according to the Kabat numbering system. However, according to Chothia (Chothia, C. and Lesk, A.M. J. Mol. Biol., 196, 901-917 (1987)), the loop equivalent to CDR-H1 extends from residue 26 to residue 32. Thus, unless indicated otherwise ‘CDR-H1’ as employed herein is intended to refer to residues 26 to 35, as described by a combination of the Kabat numbering system and Chothia’s topological loop definition. The CDRs of the light chain variable domain are located at residues 24-34 (CDR-L1), residues 50-56 (CDR-L2) and residues 89-97 (CDR-L3) according to the Kabat numbering system. Based on the alignment of sequences of different members of the immunoglobulin family, numbering schemes have been proposed and are for example described in Kabat et al., 1991, and Dondelinger et al., 2018, Frontiers in Immunology, Vol 9, article 2278.

Human immunoglobulin VH locus represents 6 main families which may be divided based on nucleotide sequence. The families and VH domains derived therefrom are generally referred to as VH1, VH2, VH3, VH4, VH5, VH6.

The term “constant domain(s)”, “constant region”, as used herein are used interchangeably to refer to the domain(s) of an antibody which is outside the variable regions. The constant domains are identical in all antibodies of the same isotype but are different from one isotype to another. Typically, the constant region of a heavy chain is formed, from N to C terminal, by CH1-hinge -CH2-CH3-optionnaly CH4, comprising three or four constant domains.

The constant region domains of the antibody molecule of the present invention, if present, may be selected having regard to the proposed function of the antibody molecule, and in particular the effector functions which may be required. For example, the constant region domains may be humanIgG1, IgG2 or IgG4 domains. In particular, human IgG constant region domains may be used, especially of the IgG1 isotype when the antibody molecule is intended for therapeutic uses and antibody effector functions are required. Alternatively, IgG2 and IgG4 isotypes may be used when the antibody molecule is intended for therapeutic purposes and antibody effector functions are not required. It will be appreciated that sequence variants of these constant region domains may also be used. For example, IgG4 molecules in which the serine at position 241 (numbered according to the Kabat numbering system) has been changed to proline as described in Angal et al. (Angal et al., 1993. A single amino acid substitution abolishes the heterogeneity of chimeric mouse/human (IgG4) antibody as observed during SDS-PAGE analysis Mol Immunol 30, 105-108) and termed IgG4P herein, may be used.

“Fc”, “Fc fragment”, “Fc region” are used interchangeably to refer to the C-terminal region of an antibody comprising the constant region of an antibody excluding the first constant region domain. Thus, Fc refers to the last two constant domains, CH2 and CH3, of IgA, IgD, and IgG, or the last three constant domains of IgE and IgM, and the flexible hinge N-terminal to these domains. The human IgG1 heavy chain Fc region is defined herein to comprise residues C226 to its carboxyl-terminus, wherein the numbering is according to the EU index as in Kabat. In the context of human IgG1, the lower hinge refers to positions 226-236, the CH2 domain refers to positions 237-340 and the CH3 domain refers to positions 341-447 according to the EU index as in Kabat. The corresponding Fc region of other immunoglobulins can be identified by sequence alignments.

The antibodies described herein are isolated. An “isolated” antibody is one which has been separated (e.g. by purification means) from a component of its natural environment.

“Multi-specific antibody” as employed herein refers to an antibody as described herein which has at least two antigen binding domains, i-e two or more antigen binding domains, for example two or three antigen binding domains, wherein the at least two antigen binding domains independently bind two different antigens or two different epitopes on the same antigen. Multi-specific antibodies may be monovalent for each specificity (antigen). Multi-specific antibodies described herein encompass monovalent and multivalent, e.g. bivalent, trivalent, tetravalent multi-specific antibodies, as well as multi-specific antibodies having different valences for different epitopes (e.g, a multi-specific antibody which is monovalent for a first antigen specificity and bivalent for a second antigen specificity which is different from the first one).

In one embodiment, the multi-specific antibody is a bi-specific antibody.

“Bispecific or Bi-specific antibody” as employed herein refers to an antibody with two antigen specificities. In one embodiment, the antibody comprises two antigen binding domains wherein one binding domain binds ANTIGEN 1 and the other binding domain binds ANTIGEN 2, i-e each binding domain is monovalent for each antigen. In one embodiment, the antibody is a tetravalent bispecific antibody, i-e the antibody comprises four antigen binding domains, wherein for example two binding domains bind ANTIGEN 1 and the other two binding domains bind ANTIGEN 2. In one embodiment, the antibody is a trivalent bispecific antibody.

In one embodiment, the multi-specific antibody is a tri-specific antibody.

“Tri-specific antibody” as employed herein refers to an antibody with three antigen binding specificities. For example, the antibody is an antibody with three antigen binding domains (trivalent), which independently bind three different antigens or three different epitopes on the same antigen, i-e each binding domain is monovalent for each antigen. In one embodiment, there are three binding domains and each of the three binding domains binds a different (distinct) antigen.

In one embodiment, there are three binding domains and two binding domains bind the same antigen, including binding the same epitope or different epitopes on the same antigen, and the third binding domain binds a different (distinct) antigen.

An antibody of the invention may be a multi-paratopic antibody.

“Multi-paratopic antibody” as employed herein refers to an antibody as described herein which comprises two or more distinct paratopes, which interact with different epitopes either from the same antigen or from two different antigens. Multi-paratopic antibodies described herein may be biparatopic, triparatopic, tetraparatopic.

“Antigen binding domain” as employed herein refers to a portion of an antibody, which comprises a part or the whole of one or more variable domains, for example a pair of variable domains VH and VL, that interact specifically with the target antigen. An antigen binding domain may comprise a single domain antibody. In one embodiment, each antigen binding domain is monovalent. Preferably each antigen binding domain comprises no more than one VH and one VL.

“Specifically” as employed herein is intended to refer to a binding domain that only recognises the antigen to which it is specific or a binding domain that has significantly higher binding affinity to the antigen to which is specific compared to affinity to antigens to which it is non-specific.

Binding affinity may be measured by standard assay, for example surface plasmon resonance, such as BIAcore.

“Protein A binding domain” as employed herein is intended to refer to a binding domain which specifically binds to protein A. A Protein A binding domain may refer to a VH3 domain or a portion of a VH3 domain which binds protein A, i-e which comprises a protein A binding interface. The portion of a VH3 domain which binds protein A does not comprise the CDRs of the VH3 domain, i-e the protein A binding interface of the VH3 does not involve the CDRs; consequently, it will be understood that a protein A binding domain does not compete with an antigen binding domain as disclosed in the present application.

In one embodiment when s is 0 and t is 0, the multi-specific antibody according to the present disclosure is provided as a dimer of a heavy and light chain of:

Formula (I) and (II) respectively, wherein the VH-CH1 portion together with the VL-C_{L} portion form a functional Fab or Fab′ fragment.

In one embodiment when s is 1 and t is 1, the multi-specific antibody according to the present disclosure is provided as a dimer of two heavy chains and two light chains of:

Formula (I) and (II) respectively, wherein the two heavy chains are connected by interchain interactions, notably at the level of CH2-CH3, and wherein the VH-CH1 portion of each heavy chain together with the VL-C_{L} portion of each light chain, form a functional Fab or Fab′ fragment. In such embodiment, the two VH-CH1- CH2- CH3 portions together with the two VL-C_{L} portions form a functional full-length antibody. In such embodiment, the full-length antibody may comprise a functional Fc region.

VH represents a heavy chain variable domain. In one embodiment VH is humanised. In one embodiment the VH is fully human.

VL represents a light chain variable domain. In one embodiment VL is humanised. In one embodiment the VL is fully human.

Generally, VH and VL pair together to form an antigen binding domain, for example in a Fab fragment. In one embodiment VH and VL form a cognate pair.

“Cognate pair” as employed herein refers to a pair of variable domains from a single antibody, which was generated in vivo, i.e. the naturally occurring pairing of the variable domains isolated from a host. A cognate pair is therefore a VH and VL pair. In one example, the cognate pair bind the antigen co-operatively.

In several instances, VH, for example when comprised in V1 and/or V2, and/or V3, may form an antigen binding domain on its own, i.e. may represent a single domain antibody which binds to an antigen of interest on its own.

VHH represents a single domain antibody which consists of a heavy chain variable domain. In one embodiment, the VHH is camelid. In one embodiment the VHH is humanised. In one embodiment the VHH is fully human.

In several instances, VL, for example when comprised in V1 and/or V2, and/or V3, may form an antigen binding domain on its own, i.e. may represent a single domain antibody which binds to an antigen of interest on its own.

“Variable region” or “variable domain” as employed herein refers to the region in an antibody chain comprising the CDRs and a framework, in particular a suitable framework.

Variable regions for use in the present disclosure will generally be derived from an antibody, which may be generated by any method known in the art.

“Derived from” as employed herein refers to the fact that the sequence employed or a sequence highly similar to the sequence employed was obtained from the original genetic material, such as the light or heavy chain of an antibody.

“Highly similar” as employed herein is intended to refer to an amino acid sequence which over its full length is 95% similar or more, such as 96, 97, 98 or 99% similar.

Variable regions for use in the present invention, as described herein above for VH and VL may be from any suitable source and may be for example, fully human or humanised.

In one embodiment, the binding domain formed by VH and VL are specific to a first antigen.

In one embodiment, the binding domain of V1 is specific to a second antigen.

In one embodiment, the binding domain of V2 is specific to a second or third antigen.

In one embodiment, the binding domain of V3 is specific to a third or fourth antigen.

In one embodiment, each one of VH-VL, V1, V2 and V3, as present, separately binds its respective antigen.

In one embodiment, the CH1 domain is a naturally occurring domain 1 from an antibody heavy chain or a derivative thereof. In one embodiment, the CH2 domain is a naturally occurring domain 2 from an antibody heavy chain or a derivative thereof. In one embodiment, the CH3 domain is a naturally occurring domain 3 from an antibody heavy chain or a derivative thereof.

In one embodiment, the C_{L} fragment, in the light chain, is a constant kappa sequence or a derivative thereof. In one embodiment, the C_{L} fragment, in the light chain, is a constant lambda sequence or a derivative thereof.

A derivative of a naturally occurring domain as employed herein is intended to refer to where at least one amino acid in a naturally occurring sequence have been replaced or deleted, for example to optimize the properties of the domain such as by eliminating undesirable properties but wherein the characterizing feature(s) of the domain is/are retained. In one embodiment, a derivative of a naturally occurring domain comprises two, three, four, five, six, seven, eight, ten, eleven or twelve amino acid substitutions or deletions compared to a naturally occurring sequence.

In one embodiment, one or more natural or engineered inter chain (i.e. inter light and heavy chain) disulphide bonds are present in the functional Fab or Fab′ fragment.

In one embodiment, a “natural” disulfide bond is present between a CH1 and C_{L} in the polypeptide chains of Formula (I) and (II).

When the C_{L} domain is derived from either Kappa or Lambda, the natural position for a bond forming cysteine is 214 in human cKappa and cLambda (Kabat numbering 4^{th} edition 1987).

The exact location of the disulfide bond forming cysteine in CH1 depends on the particular domain actually employed. Thus, for example in human gamma-1 the natural position of the disulfide bond is located at position 233 (Kabat numbering 4^{th} edition 1987). The position of the bond forming cysteine for other human isotypes such as gamma 2, 3, 4, IgM and IgD are known, for example position 127 for human IgM, IgE, IgG2, IgG3, IgG4 and 128 of the heavy chain of human IgD and IgA2B.

Optionally, there may be a disulfide bond between the VH and VL of the polypeptides of formula I and II.

In one embodiment, the multi-specific antibody according to the disclosure has a disulfide bond in a position equivalent or corresponding to that naturally occurring between CH1 and C_{L}.

In one embodiment, a constant region comprising CH1 and a constant region such as C_{L} has a disulfide bond which is in a non-naturally occurring position. This may be engineered into the molecule by introducing cysteine(s) into the amino acid chain at the position or positions required. This non-natural disulfide bond is in addition to or as an alternative to the natural disulfide bond present between CH1 and C_{L}. The cysteine(s) in natural positions can be replaced by an amino acid such as serine which is incapable on forming a disulfide bridge.

Introduction of engineered cysteines can be performed using any method known in the art. These methods include, but are not limited to, PCR extension overlap mutagenesis, site-directed mutagenesis or cassette mutagenesis (see, generally, Sambrook et al., Molecular Cloning, A Laboratory Manual, Cold Spring Harbour Laboratory Press, Cold Spring Harbour, NY, 1989; Ausbel et al., Current Protocols in Molecular Biology, Greene Publishing & Wiley-Interscience, NY, 1993). Site-directed mutagenesis kits are commercially available, e.g. QuikChange® Site-Directed Mutagenesis kit (Stratagene, La Jolla, CA). Cassette mutagenesis can be performed based on Wells et al., 1985, Gene, 34:315-323. Alternatively, mutants can be made by total gene synthesis by annealing, ligation and PCR amplification and cloning of overlapping oligonucleotides.

In one embodiment, a disulfide bond between CH1 and C_{L} is completely absent, for example the interchain cysteines may be replaced by another amino acid, such as serine. Thus, in one embodiment there are no interchain disulphide bonds in the functional Fab fragment of the molecule. Disclosures such as WO2005/003170, incorporated herein by reference, describe how to provide Fab fragments without an inter chain disulphide bond.

Preferred antibody formats for use in the present invention include appended IgG and appended Fab, wherein a whole IgG or a Fab fragment, respectively, is engineered by appending at least one additional antigen-binding domain (e.g. one, two, three or four additional antigen-binding domains), for example a single domain antibody (such as VH or VL, or VHH), a scFv, a dsscFv, a dsFv to the N- and/or C-terminus of the light chain of said IgG or Fab, and optionally to the heavy chain of said IgG or Fab, for example as described in WO2009/040562, WO2010035012, WO2011/030107, WO2011/061492, WO2011/061246 and WO2011/086091 all incorporated herein by reference. In particular, the Fab-Fv format was first disclosed in WO2009/040562 and the disulphide stabilized version thereof, the Fab-dsFv, was first disclosed in WO2010/035012. A single linker Fab-dsFv, wherein the dsFv is connected to the Fab via a single linker between either the VL or VH domain of the Fv, and the C terminal of the LC of the Fab, was first disclosed in WO2014/096390, incorporated herein by reference. An appended IgG comprising a full-length IgG engineered by appending a dsFv to the C-terminus of the light chain (and optionally to the heavy chain) of the IgG, was first disclosed in WO2015/197789, incorporated herein by reference.

Another preferred antibody format for use in the present invention comprises a Fab linked to two scFvs or dsscFvs, each scFv or dsscFv binding the same or a different target (e.g., one scFv or dsscFv binding a therapeutic target and one scFv or dsscFv that increases half-life by binding, for instance, albumin). Such antibody fragments are described in International Patent Application Publication No WO2015/197772, which is hereby incorporated by reference in its entirety and particularly with respect to the discussion of antibody fragments. Another preferred antibody for use in the present invention fragment comprises a Fab linked to only one scFv or dsscFv, as described for example in WO2013/068571 incorporated herein by reference, and Dave et al., 2016, Mabs, 8(7) 1319-1335.

V1, when present, represents a dsscFv, a dsFv, a scFv, a VH, a VL or a VHH, for example a dsscFv, a dsFv, or a scFv.

V2, when present, represents a dsscFv, a dsFv, a scFv, a VH, a VL or a VHH, for example a dsscFv, a dsFv, or a scFv.

V3, when present, represents a dsscFv, a dsFv, a scFv, a VH, a VL or a VHH, for example a dsscFv, a dsFv, or a scFv.

The polypeptide chain of formula (II) comprises at least one dsscFv, dsFv, scFv, VH, or VHH.

“Single chain variable fragment” or “scFv” as employed herein refers to a single chain variable fragment comprising or consisting of a heavy chain variable domain (VH) and a light chain variable domain (VL) which is stabilised by a peptide linker between the VH and VL variable domains. The VH and VL variable domains may be in any suitable orientation, for example the C-terminus of VH may be linked to the N-terminus of VL or the C-terminus of VL may be linked to the N-terminus of VH.

“Disulphide-stabilised single chain variable fragment” or “dsscFv” as employed herein refers to a single chain variable fragment which is stabilised by a peptide linker between the VH and VL variable domain and also includes an inter-domain disulphide bond between VH and VL.

“Disulphide-stabilised variable fragment” or “dsFv” as employed herein refers to a single chain variable fragment which does not include a peptide linker between the VH and VL variable domains and is instead stabilised by an interdomain disulphide bond between VH and VL.

In one embodiment, when V1 and/or V2 and/or V3 are a dsFv or a dsscFv, the disulfide bond between the variable domains VH and VL of V1 and/or V2 and/or V3 is between two of the residues listed below (unless the context indicates otherwise Kabat numbering is employed in the list below). Wherever reference is made to Kabat numbering the relevant reference is Kabat et al., 1991 (5^{th} edition, Bethesda, Md.), in Sequences of Proteins of Immunological Interest, US Department of Health and Human Services, NIH, USA.

In one embodiment the disulfide bond is in a position selected from the group comprising:
VH37 + VL95C see for example Protein Science 6, 781-788 Zhu et al (1997);
VH44 + VL100 see for example; for example, Weatherill et al., Protein Engineering, Design & Selection, 25 (321-329), 2012);
VH44 + VL105 see for example J Biochem. 118, 825-831 Luo et al (1995);
VH45 + VL87 see for example Protein Science 6, 781-788 Zhu et al (1997);
VH55 + VL101 see for example FEBS Letters 377 135-139 Young et al (1995);
VH100 + VL50 see for example Biochemistry 29 1362-1367 Glockshuber et al (1990);
VH100b + VL49; see for example Biochemistry 29 1362-1367 Glockshuber et al (1990);
VH98 + VL 46; see for example Protein Science 6, 781-788 Zhu et al (1997);
VH101 + VL46; see for example Protein Science 6, 781-788 Zhu et al (1997);
VH105 + VL43 see for example; Proc. Natl. Acad. Sci. USA Vol. 90 pp.7538-7542 Brinkmann et al (1993); or Proteins 19, 35-47 Jung et al (1994),
VH106 + VL57 see for example FEBS Letters 377 135-139 Young et al (1995) and a position corresponding thereto in variable region pair located in the molecule.

In one embodiment, the disulphide bond is formed between positions VH44 and VL100.

The amino acid pairs listed above are in the positions conducive to replacement by cysteines such that disulfide bonds can be formed. Cysteines can be engineered into these desired positions by known techniques. In one embodiment, therefore, an engineered cysteine according to the present disclosure refers to where the naturally occurring residue at a given amino acid position has been replaced with a cysteine residue.

Introduction of engineered cysteines can be performed using any method known in the art. These methods include, but are not limited to, PCR extension overlap mutagenesis, site-directed mutagenesis or cassette mutagenesis (see, generally, Sambrook et al., Molecular Cloning, A Laboratory Manual, Cold Spring Harbour Laboratory Press, Cold Spring Harbour, NY, 1989; Ausbel et al., Current Protocols in Molecular Biology, Greene Publishing & Wiley-Interscience, NY, 1993). Site-directed mutagenesis kits are commercially available, e.g. QuikChange® Site-Directed Mutagenesis kit (Stratagen, La Jolla, CA). Cassette mutagenesis can be performed based on Wells et al., 1985, Gene, 34:315-323. Alternatively, mutants can be made by total gene synthesis by annealing, ligation and PCR amplification and cloning of overlapping oligonucleotides.

Accordingly, in one embodiment when V1 and/or V2 and/or V3 are a dsFv or a dsscFv, the variable domains VH and VL of V1 and/or the variable domains VH and VL of V2, and/or the variable domains VH and VL of V3, may be linked by a disulfide bond between two cysteine residues, wherein the position of the pair of cysteine residues is selected from the group consisting of: VH37 and VL95, VH44 and VL100, VH44 and VL105, VH45 and VL87, VH100 and VL50, VH100b and VL49, VH98 and VL46, VH101 and VL46, VH105 and VL43 and VH106 and VL57.

In one embodiment when V1 and/or V2 and/or V3 are a dsFv or a dsscFv, the variable domains VH and VL of V1 and/or the variable domains VH and VL of V2,and/or the variable domains VH and VL of V3, may be linked by a disulfide bond between two cysteine residues, one in VH and one in VL, which are outside of the CDRs wherein the position of the pair of cysteine residues is selected from the group consisting of VH37 and VL95, VH44 and VL100, VH44 and VL105, VH45 and VL87, VH100 and VL50, VH98 and VL46, VH105 and VL43 and VH106 and VL57.

In one embodiment when V1 is a dsFv or a dsscFv, the variable domains VH and VL of V1 are linked by a disulphide bond between two engineered cysteine residues, one at position VH44 and the other at VL100. In one embodiment when V2 is a dsFv or a dsscFv, the variable domains VH and VL of V2 are linked by a disulphide bond between two engineered cysteine residues, one at position VH44 and the other at VL100. In one embodiment when V3 is a dsFv or a dsscFv, the variable domains VH and VL of V3 are linked by a disulphide bond between two engineered cysteine residues, one at position VH44 and the other at VL100.

In one embodiment when V1 is a dsscFv, a dsFv, or a scFv, the VH domain of V1 is attached to X. In one embodiment when V1 is a dsscFv, a dsFv, or a scFv, the VL domain of V1 is attached to X. In one embodiment when V2 is a dsscFv, a dsFv, or a scFv, the VH domain of V2 is attached to Y. In one embodiment when V2 is a dsscFv, a dsFv, or a scFv, the VL domain of V2 is attached to Y. In one embodiment when V3 is a dsscFv, a dsFv, or a scFv, the VH domain of V3 is attached to Z. In one embodiment when V3 is a dsscFv, a dsFv, or a scFv, the VL domain of V3 is attached to Z.

The skilled person will appreciate that when V1 and/or V2 and/or V3 represents a dsFv, the multi-specific antibody will comprise a third polypeptide encoding the corresponding free VH or VL domain which is not attached to X or Y or Z. When V1 and V2, V2 and V3, or V1 and V2 and V3 are a dsFv then the “free variable domain” (i.e. the domain linked to via a disulphide bond to the remainder of the polypeptide) will be common to both chains. Thus, whilst the actual variable domain fused or linked via X or Y or Z to the polypeptide may be different in each polypeptide chain, the free variable domains paired therewith will generally be identical to each other.

In one embodiment, V1 is a VH, a VL or a VHH, which forms an antigen binding domain. In one embodiment, V1 is a VH which binds to an antigen of interest co-operatively with a complementary VL. In one embodiment, V1 is a VL which binds to an antigen of interest co-operatively with a complementary VH.

In one embodiment, V2 is a VH, a VL or a VHH, which forms an antigen binding domain. In one embodiment, V2 is a VH which binds to an antigen of interest co-operatively with a complementary VL. In one embodiment, V2 is a VL which binds to an antigen of interest co-operatively with a complementary VH.

In one embodiment, V3 is a VH, a VL or a VHH, which forms an antigen binding domain. In one embodiment, V3 is a VH which binds to an antigen of interest co-operatively with a complementary VL. In one embodiment, V3 is a VL which binds to an antigen of interest co-operatively with a complementary VH.

In one embodiment, V1 is a VH, V2 is a VL which is complementary to the VH of V1, and VH/VL, i.e. V1/V2, pair to form an antigen binding domain, i.e. the VH of V1 binds to an antigen of interest co-operatively with a complementary VL of V2.

In one embodiment, V1 is a VL, V2 is a VH which is complementary to the VL of V1, and VL/VH, i.e. V1/V2, pair to form an antigen binding domain, i.e. the VL of V1 binds to an antigen of interest co-operatively with a complementary VH of V2.

In one embodiment when V1 is a VH and V2 is a complementary VL, the variable domains VH of V1 and VL of V2 may be linked by a disulphide bond between two engineered cysteine residues, one at position VH44 of V1 and the other at VL100 of V2. In one embodiment when V1 is a VL and V2 is a complementary VH, the variable domains VL of V1 and VH of V2 may be linked by a disulphide bond between two engineered cysteine residues, one at position VL100 of V1 and the other at position VH44 of V2.

The polypeptide chain of formula (I) of the present disclosure comprises a protein A binding domain. In one embodiment, the polypeptide chain of formula (I) comprises one, two or three protein A binding domains.

Protein A is a 42 kDa surface protein originally found in the cell wall of the bacteria Staphylococcus aureus. Protein A has been widely used to detect, quantify and purify immunoglobulins. Protein A has been reported to bind the Fab portion derived from the VH3 family antibodies, and the Fc gamma region in the constant region portion of IgG (between the CH2 and CH3 domains). The crystal structure of the complex formed by protein A and the Fab has been described for example in Graille et al., 2000, PNAS, 97(10): 5399-5404.

In the context of the present disclosure, protein A encompasses natural protein A and any variant or derivative thereof, to the extent that the protein A variant or derivative maintains its ability to bind VH3 domains.

In one embodiment, the polypeptide chain of formula (I) comprises a protein A binding domain which is present in VH and/or CH2-CH3 and/or V1. In one embodiment, the polypeptide chain of formula (I) comprises one, two or three protein A binding domains, which is/are present in VH and/or CH2-CH3 and/or V1. In one embodiment, the polypeptide chain of formula (I) comprises only one protein A binding domain which is present in VH or V1. In one embodiment, s is 0, t is 0 and the polypeptide chain of formula (I) comprises only one protein A binding domain which is present in VH or V1. In one embodiment, the polypeptide chain of formula (I) comprises only one protein A binding domain which is present in VH. In one embodiment, s is 0, t is 0, p is 0, and the polypeptide chain of formula (I) comprises only one protein A binding domain which is present in VH. In one embodiment, the polypeptide chain of formula (I) comprises only one protein A binding domain which is present in V1. In one embodiment, s is 0, t is 0, p is 1, and the polypeptide chain of formula (I) comprises only one protein A binding domain which is present in V1.

In one embodiment, the polypeptide chain of formula (I) comprises two protein A binding domains. In one embodiment, the polypeptide chain of formula (I) comprises two protein A binding domains which are present in VH and CH2-CH3 respectively. In another embodiment, the polypeptide chain of formula (I) comprises two protein A binding domains which are present in VH and V1 respectively. In another embodiment, the polypeptide chain of formula (I) comprises two protein A binding domains which are present in CH2-CH3 and V1 respectively.

In one embodiment, the polypeptide chain of formula (I) comprises three protein A binding domains, each one being present in VH, CH2-CH3 and V1.

Natural protein A can interact in particular with the Fc gamma region, in the constant region portion of IgG. More particularly, protein A can interact with a binding domain between the CH2 and the CH3. In one embodiment when s is 1, t is 1, both CH2 and CH3 are naturally occurring domains of the IgG class.

In some embodiments, the protein A binding domain(s) comprise(s) or consist(s) of a VH3 domain or variant thereof which binds protein A. In some embodiments, the protein A binding domain(s) comprise(s) or consist(s) of a naturally occurring VH3 domain. In some embodiments, a variant of a VH3 domain which binds protein A is a variant of a naturally occurring VH3 domain, said naturally occurring VH3 domain being unable to bind protein A.

The polypeptide chain of formula (II) comprises at least one dsscFv, dsFv, scFv, VH, or VHH. In one embodiment, the polypeptide chain of formula (II) comprises only one dsscFv. In one embodiment, the polypeptide chain of formula (II) comprises only one dsFv. In one embodiment, the polypeptide chain of formula (II) comprises only one scFv. In one embodiment, the polypeptide chain of formula (II) comprises only one VH. In one embodiment, the polypeptide chain of formula (II) comprises only one VHH.

The polypeptide chain of formula (II) of the present disclosure does not bind protein A. In one embodiment, the binding domain of V2 does not bind protein A. In one embodiment, the binding domain of V3 does not bind protein A. In one embodiment, both V2 and V3 do not bind protein A.

In some embodiments, V2 and/or V3 comprise(s) or consist(s) of a VH1 and/or a VH2 and/or a VH4 and/or a VH5 and/or a VH6 and do(es) not comprise a VH3 domain. In some embodiments, V2 and/or V3, comprise(s) or consist(s) of a VH3 domain or variant thereof which does not bind protein A. In some embodiments, V2 and/or V3, comprise(s) or consist(s) of a naturally occurring VH3 domain being unable to bind protein A. In some embodiments, a variant of a VH3 domain which does not bind protein A is a variant of a naturally occurring VH3, said naturally occurring VH3 domain being able to bind protein A.

Human VH3 germline genes and VH3 domains (or frameworks) have been well characterized. Many of the naturally occurring VH3 domains have the capacity to bind protein A but certain naturally occurring VH3 domains do not have the capacity to bind protein A (see Roben et al., 1995, J Immunol.;154(12):6437-6445).

A VH3 domain for use in the present disclosure can be obtained by several methods. In one embodiment, a VH3 domain for use in the present disclosure is a naturally occurring VH3 domain, selected for its ability or inability to bind protein A, depending on its position within the polypeptide (I) and/or (II) of the disclosure. For example, a panel of antibodies may be generated against an antigen of interest by immunisation of a non-human animal, then humanised, and the humanised antibodies may be screened and selected based on their ability or inability to bind protein A via the humanised VH3 domain, for example against a protein A affinity column. Alternatively, display technologies (e.g. phage display, yeast display, ribosome display, bacterial display, mammalian cell surface display, mRNA display, DNA display) may be used to screen antibody libraries and select antibodies comprising a VH3 domain which binds, notably via a protein A binding interface which does not involve the CDRs, or does not bind protein A.

Alternatively, a VH3 domain for use in the present disclosure is a variant of a naturally occurring VH3. In one embodiment, a VH3 variant comprises a sequence of a naturally occurring VH3 able to bind protein A, and further comprising at least one amino acid mutation, which abolishes its ability to bind protein A. In one embodiment, a VH3 variant which binds protein A comprises a sequence of a naturally occurring VH3 unable to bind protein A, and further comprises at least one amino acid mutation. In such embodiment, the mutation(s) is/are responsible for the VH3 domain to gain the ability to bind protein A, i-e the mutation(s) contribute(s) to the generation of a protein A binding domain which was not naturally present.

In one embodiment, a VH3 variant comprises 1, 2, 3, 4, 5, 6, 7, 8, 9, 10, 11, or 12 amino acid mutations. In one embodiment, a VH3 variant comprises a mutation at the position 15, 17, 19, 57, 59, 64, 65, 66, 68, 70, 81 or 82 on the VH3, numbering according to Kabat and as described for example in Graille et al., 2000, PNAS, 97(10): 5399-5404). More particularly, a VH3 variant may comprise a mutation at the position 82a or 82b on the VH3, numbering according to Kabat and as described for example in Graille et al., 2000, PNAS, 97(10): 5399-5404). The mutation may be a substitution, a deletion, or an insertion. In one embodiment, the VH3 variant comprises a substitution at the position 15, 17, 19, 57, 59, 64, 65, 66, 68, 70, 81 or 82 on the VH3, numbering according to Kabat. More particularly, the VH3 variant may comprise a substitution at the position 82a or 82b on the VH3, numbering according to Kabat and as described for example in Graille et al., 2000, PNAS, 97(10): 5399-5404).

Naturally occurring VH1, VH2, VH4, VH5 and VH6 do not bind protein A. In one embodiment, a VH domain which does not bind protein A is a VH1. In one embodiment, a VH domain which does not bind protein A is a VH2. In one embodiment, a VH domain which does not bind protein A is a VH4. In one embodiment, a VH domain which does not bind protein A is a VH5. In one embodiment, a VH domain which does not bind protein A is a VH6.

In the context of the invention, new methods have been developed which may be used for assessing the binding of a polypeptide or binding domain according to the invention, to protein A. A Protein-A interaction assay has been developed to qualitatively assess binding to protein A. Therefore, in one aspect, the invention provides a method for selecting a polypeptide or binding domain according to the invention, said method comprising the use of a Protein-A interaction assay. A Protein-A interaction assay as described in the Examples may be used.

In one aspect, the invention provides a method for selecting a dsscFv, a dsFv, a scFv, a VH, or a VHH for use in the polypeptide (II) according to the invention, i.e. which does not bind Protein A, said method comprising:
a) producing a test molecule comprising a Fab which does not bind protein A, appended with a a dsscFv, a dsFv, a scFv, a VH, or a VHH; and
b) loading the test molecule obtained at step a) onto a Protein A chromatography column; and,
c) recovering the Flow Through obtained from step b); and,
d) washing the column of step b) with a running buffer; and,
e) performing an acidic step elution; and,
f) selecting a dsscFv, a dsFv, a scFv, a VH, or a VHH which is comprised in a test molecule recovered from the Flow Through.

In one embodiment, the Fab which does not bind protein A is a murine Fab. In one embodiment, the Protein A chromatography column is POROS™ A 20 µm Column (Thermo Fisher Scientific, Waltham, MA). In one embodiment, the running buffer is PBS pH 7.4. In one embodiment, at step d) the column is washed over 60 column volumes for 30 minutes. In one embodiment, the acidic step elution at step e) is performed with 0.1 M Glycine-HCl pH 2.7 at 2.0 ml/min, for 2 minutes.

In addition, a Surface Plasmon resonance assay using Biacore has been developed to quantitatively assess binding to protein A. Therefore, in one aspect, the invention provides a method for selecting a polypeptide or binding domain according to the invention, said method comprising the use of a Biacore assay. A Biacore assay as described in the Examples may be used.

In one aspect, the invention provides a method for selecting a dsscFv, a dsFv, a scFv, a VH, or a VHH for use in the polypeptide (II) according to the invention, i.e. which does not bind Protein A, said method comprising:
a) producing a test molecule comprising a Fab which does not bind protein A, appended with a dsscFv, a dsFv, a scFv, a VH, or a VHH; and,
b) measuring the binding of the test molecule obtained at step a) by Surface Plasmon resonance, for example using Biacore; and,
c) titrating a non-binding negative control; and,
d) selecting a dsscFv, a dsFv, a scFv, a VH, or a VHH which is comprised in a test molecule that has a binding response that is no greater than 2-fold higher than the response observed for the non-binding negative control.

In one embodiment, the Fab which does not bind protein A is a murine Fab.

As described in the Examples, the inventors showed the importance of completely abolishing the ability of the antibody light chain, i.e. the polypeptide chain of formula (II) to bind protein A in the context of the invention, while the polypeptide chain of formula (I) binds to protein A. This method therefore allows identification of polypeptides or protein A binding domains having a strong binding to protein A, which could be selected and used as part of the polypeptide (I), and polypeptides or protein A binding domains having a weak binding to protein A, which should not be comprised in the polypeptide chain of formula (II).

In some embodiments, p is 1. In some embodiments, p is 0. In some embodiments, q is 1. In some embodiments, q is 0, and r is 1. In some embodiments, r is 1. In some embodiments, q is 1 and r is 0.

In some embodiments, q is 1 and r is 1. In some embodiments, s is 1. In some embodiments, s is 0. In some embodiments, t is 1. In some embodiments, t is 0. In some embodiments, s is 1 and t is 1. In some embodiments, s is 0 and t is 0.

In one embodiment, p is 1, q is 1, r is 0, s is 0 and t is 0, and V1 and V2 both represent a dsscFv . Thus, in one aspect, there is provided a multi-specific antibody comprising or consisting of:
a) a polypeptide chain of formula (Ia):

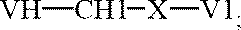

and
b) a polypeptide chain of formula (IIa):

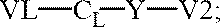

wherein:
   VH represents a heavy chain variable domain;
   CH1 represents domain 1 of a heavy chain constant region;
   X represents a bond or linker;
   Y represents a bond or linker;
   V1 represents a dsscFv;
   VL represents a light chain variable domain;
   C_{L} represents a domain from a light chain constant region, such as Ckappa;
   V2 represents a dsscFv;
wherein the polypeptide chain of formula (Ia) comprises a protein A binding domain; and wherein the polypeptide chain of formula (IIa) does not bind protein A.

In such embodiment, V2 does not bind protein A, i-e the dsscFv of V2 does not comprise a protein A binding domain. In one embodiment, V2, i-e the dsscFv of V2, comprises a VH1 domain. In another embodiment, V2, i-e the dsscFv of V2, comprises a VH3 domain which does not bind protein A. In one embodiment, V2, i-e the dsscFv of V2, comprises a VH2 domain. In one embodiment, V2, i-e the dsscFv of V2, comprises a VH4 domain. In one embodiment, V2, i-e the dsscFv of V2, comprises a VH5 domain. In one embodiment, V2, i-e the dsscFv of V2, comprises a VH6 domain. In one embodiment, the polypeptide chain of formula (Ia) comprises only one protein A binding domain present in VH or V1. In one embodiment, the polypeptide chain of formula (Ia) comprises only one protein A binding domain present in V1. In another embodiment, the polypeptide chain of formula (Ia) comprises two protein A binding domains present in VH and V1 respectively.

In another embodiment, p is 0, q is 1, r is 0, s is 1, t is 1, and V2 is a dsscFv. Thus, in one aspect, there is provided a multi-specific antibody comprising or consisting of:
a) a polypeptide chain of formula (Ib):

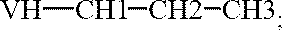

and
b) a polypeptide chain of formula (IIb):

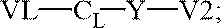

wherein:
   VH represents a heavy chain variable domain;
   CH1 represents domain 1 of a heavy chain constant region;
   CH2 represents domain 2 of a heavy chain constant region;
   CH3 represents domain 3 of a heavy chain constant region;
   Y represents a bond or linker;
   VL represents a light chain variable domain;
   C_{L} represents a domain from a light chain constant region, such as Ckappa;
   V2 represents a dsscFv;
   wherein the polypeptide chain of formula (Ib) comprises a protein A binding domain; and wherein the polypeptide chain of formula (IIb) does not bind protein A.

In such embodiment, V2 does not bind protein A, i-e the dsscFv of V2 does not comprise a protein A binding domain. In one embodiment, V2, i-e the dsscFv of V2, comprises a VH1 domain. In another embodiment, V2, i-e the dsscFv of V2, comprises a VH3 domain which does not bind protein A. In one embodiment, the polypeptide chain of formula (Ib) comprises only one protein A binding domain present in VH or CH2-CH3. In another embodiment, the polypeptide chain of formula (Ib) comprises two protein A binding domains present in VH and CH2-CH3 respectively.

In another embodiment, p is 0, q is 1, r is 0, s is 1, t is 1, and V2 is a dsFv. Thus, in one aspect, there is provided a multi-specific antibody comprising or consisting of:
a) a polypeptide chain of

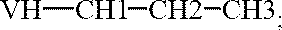

and
b) a polypeptide chain of

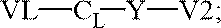

wherein:
   VH represents a heavy chain variable domain;
   CH1 represents domain 1 of a heavy chain constant region;
   CH2 represents domain 2 of a heavy chain constant region;
   CH3 represents domain 3 of a heavy chain constant region;
   Y represents a bond or linker;
   VL represents a light chain variable domain;
   C_{L} represents a domain from a light chain constant region, such as Ckappa;
   V2 represents a dsFv;
   wherein the polypeptide chain of formula (Ic) comprises a protein A binding domain; and wherein the polypeptide chain of formula (IIc) does not bind protein A.

In such embodiment, V2, i-e the dsFv of V2, does not bind protein A. In one embodiment, V2, i-e the dsFv of V2, comprises a VH1 domain. In another embodiment, V2, i-e the dsFv of V2, comprises a VH3 domain which does not bind protein A. In one embodiment, the polypeptide chain of formula (Ic) comprises only one protein A binding domain present in VH or CH2-CH3. In another embodiment, the polypeptide chain of formula (Ic) comprises two protein A binding domains present in VH and CH2-CH3 respectively.

In another embodiment, p is 0, q is 0, r is 1, s is 1, t is 1, and V3 is a dsscFv. Thus, in one aspect, there is provided a multi-specific antibody comprising or consisting of:
a) a polypeptide chain of

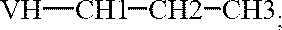

and
b) a polypeptide chain of

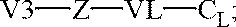

wherein:
   VH represents a heavy chain variable domain;
   CH1 represents domain 1 of a heavy chain constant region;
   CH2 represents domain 2 of a heavy chain constant region;
   CH3 represents domain 3 of a heavy chain constant region;
   Z represents a bond or linker;
   VL represents a light chain variable domain;
   C_{L} represents a domain from a light chain constant region, such as Ckappa;
   V3 represents a dsscFv;
   wherein the polypeptide chain of formula (Id) comprises a protein A binding domain; and wherein the polypeptide chain of formula (IId) does not bind protein A.

In such embodiment, V3, i-e the dsscFv of V3, does not bind protein A. In one embodiment, V3, i-e the dsscFv of V3, comprises a VH1 domain. In another embodiment, V3 i-e the dsscFv of V3, comprises a VH3 domain which does not bind protein A. In one embodiment, the polypeptide chain of formula (Id) comprises only one protein A binding domain present in VH or CH2-CH3. In another embodiment, the polypeptide chain of formula (Id) comprises two protein A binding domains present in VH and CH2-CH3 respectively.

In one embodiment, X is a bond.

In one embodiment, Y is a bond.

In one embodiment, Z is a bond.

In one embodiment, both X and Y are bonds. In one embodiment, both X and Z are bonds. In one embodiment, both Y and Z are bonds. In one embodiment, X, Y and Z are bonds.

In one embodiment, X is a linker, preferably a peptide linker, for example a suitable peptide for connecting the portions CH1 and V1 when s is 0 and t is 0, or for example for connecting the portions CH3 and V1 when t is 1.

In one embodiment, Y is a linker, preferably a peptide linker, for example a suitable peptide for connecting the portions C_{L} and V2.

In one embodiment, Z is a linker, preferably a peptide linker, for example a suitable peptide for connecting the portions VL and V3.

In one embodiment, both X and Y are linkers. In one embodiment, both X and Y are peptide linkers. In one embodiment, both X and Z are linkers. In one embodiment, both X and Z are peptide linkers. In one embodiment both Y and Z are linkers. In one embodiment both Y and Z are peptide linkers. In one embodiment, X, Y and Z are linkers. In one embodiment, X, Y and Z are peptide linkers.

The term “peptide linker” as used herein refers to a peptide comprised of amino acids. A range of suitable peptide linkers will be known to the person of skill in the art.

In one embodiment, the peptide linker is 50 amino acids in length or less, for example 25 amino acids or less, such as 20 amino acids or less, such as 15 amino acids or less, such as 5, 6, 7, 8, 9, 10, 11, 12, 13 or 14 amino acids in length.

In one embodiment, the linker is selected from a sequence shown in sequence 1 to 67.

In one embodiment, the linker is selected from a sequence shown in SEQ ID NO: 1 or SEQ ID NO: 2.

In one embodiment, X has the sequence SGGGGTGGGGS (SEQ ID NO: 1). In one embodiment, Y has the sequence SGGGGTGGGGS (SEQ ID NO: 1). In one embodiment, Z has the sequence SGGGGTGGGGS (SEQ ID NO: 1). In one embodiment, X has the sequence SGGGGSGGGGS (SEQ ID NO: 2). In one embodiment, Y has the sequence SGGGGSGGGGS (SEQ ID NO: 2). In one embodiment, Z has the sequence SGGGGSGGGGS (SEQ ID NO: 2). In one embodiment when p is 1, q is 1, r is 0 and Z is absent, X has the sequence given in SEQ ID NO:1 and Y has the sequence given in SEQ ID NO:2.

In one embodiment, X has the sequence given in SEQ ID NO:69 or 70. In one embodiment, Y has the sequence given in SEQ ID NO: 69 or 70. In one embodiment, Z has the sequence given in SEQ ID NO: 69 or 70. In one embodiment when p is 1, q is 1, r is 0 and Z is absent, X has the sequence given in SEQ ID NO:69 and Y has the sequence given in SEQ ID NO:70.

TABLE 1

Hinge linker sequences

| SEQ ID NO: | SEQUENCE |
|---|---|
| 3 | DKTHTCAA |
| 4 | DKTHTCPPCPA |
| 5 | DKTHTCPPCPATCPPCPA |
| 6 | DKTHTCPPCPATCPPCPATCPPCPA |
| 7 | DKTHTCPPCPAGKPTLYNSLVMSDTAGTCY |
| 8 | DKTHTCPPCPAGKPTHVNVSVVMAEVDGTCY |
| 9 | DKTHTCCVECPPCPA |
| 10 | DKTHTCPRCPEPKSCDTPPPCPRCPA |
| 11 | DKTHTCPSCPA |

TABLE 2

Flexible linker sequences

| SEQ ID NO: | SEQUENCE |
|---|---|
| 12 | SGGGGSE |
| 13 | DKTHTS |
| 14 | (S)GGGGS |
| 15 | (S)GGGGSGGGGS |
| 16 | (S)GGGGSGGGGSGGGGS |
| 17 | (S)GGGGSGGGGSGGGGSGGGGS |
| 18 | (S)GGGGSGGGGSGGGGSGGGGSGGGGS |
| 19 | AAAGSG-GASAS |
| 20 | AAAGSG-XGGGS-GASAS |
| 21 | AAAGSG-XGGGSXGGGS -GASAS |
| 22 | AAAGSG- XGGGSXGGGSXGGGS -GASAS |
| 23 | AAAGSG- XGGGSXGGGSXGGGSXGGGS-GASAS |
| 24 | AAAGSG-XS-GASAS |
| 25 | PGGNRGTTTTRRPATTTGSSPGPTQSHY |
| 26 | ATTTGSSPGPT |
| 27 | ATTTGS |
| - | GS |
| 28 | EPSGPISTINSPPSKESHKSP |
| 29 | GTVAAPSVFIFPPSD |
| 30 | GGGGIAPSMVGGGGS |
| 31 | GGGGKVEGAGGGGGS |
| 32 | GGGGSMKSHDGGGGS |
| 33 | GGGGNLITIVGGGGS |
| 34 | GGGGVVPSLPGGGGS |
| 35 | GGEKSIPGGGGS |
| 36 | RPLSYRPPFPFGFPSVRP |
| 37 | YPRSIYIRRRHPSPSLTT |
| 38 | TPSHLSHILPSFGLPTFN |
| 39 | RPVSPFTFPRLSNSWLPA |
| 40 | SPAAHFPRSIPRPGPIRT |
| 41 | APGPSAPSHRSLPSRAFG |
| 42 | PRNSIHFLHPLLVAPLGA |
| 43 | MPSLSGVLQVRYLSPPDL |
| 44 | SPQYPSPLTLTLPPHPSL |
| 45 | NPSLNPPSYLHRAPSRIS |
| 46 | LPWRTSLLPSLPLRRRP |
| 47 | PPLFAKGPVGLLSRSFPP |
| 48 | VPPAPVVSLRSAHARPPY |
| 49 | LRPTPPRVRSYTCCPTP- |
| 50 | PNVAHVLPLLTVPWDNLR |
| 51 | CNPLLPLCARSPAVRTFP |

(S) is optional in sequences 14 to 18.

Examples of rigid linkers include the peptide sequences GAPAPAAPAPA (SEQ ID NO: 52), PPPP (SEQ ID NO: 53) and PPP.

In one embodiment, the peptide linker is an albumin binding peptide.

Examples of albumin binding peptides are provided in WO2007/106120 and include:

TABLE 3

| SEQ ID NO: | SEQUENCE |
|---|---|
| 54 | DLCLRDWGCLW |
| 55 | DICLPRWGCLW |
| 56 | MEDICLPRWGCLWGD |
| 57 | QRLMEDICLPRWGCLWEDDE |
| 58 | QGLIGDICLPRWGCLWGRSV |
| 59 | QGLIGDICLPRWGCLWGRSVK |
| 60 | EDICLPRWGCLWEDD |
| 61 | RLMEDICLPRWGCLWEDD |
| 62 | MEDICLPRWGCLWEDD |
| 63 | MEDICLPRWGCLWED |
| 64 | RLMEDICLARWGCLWEDD |
| 65 | EVRSFCTRWPAEKSCKPLRG |
| 66 | RAPESFVCYWETICFERSEQ |
| 67 | EMCYFPGICWM |

Advantageously, use of albumin binding peptides as a linker may increase the half-life of the multi-specific antibody.

In one embodiment, when V1 is a scFv or a dsscFv, there is a linker for example a suitable peptide linker for connecting the variable domains VH and VL of V1.

In one embodiment, when V2 is a scFv or a dsscFv, there is a linker for example a suitable peptide linker for connecting the variable domains VH and VL of V2.

In one embodiment, when V3 is a scFv or a dsscFv, there is a linker for example a suitable peptide linker for connecting the variable domains VH and VL of V3.

In one embodiment, the peptide linker in the scFv or dsscFv is in range from 12 to 25 amino acids in length, such as 15 to 20 amino acids. In one embodiment, the peptide linker in the scFv or dsscFv is 15, 16, 17, 18, 19, 20, 21, 22, 23, 24 or 25 amino acids.

In one embodiment when V1 is a scFv or a dsscFv, the linker connecting the variable domains VH and VL of V1 has the sequence GGGGSGGGGSGGGGSGGGGS (SEQ ID NO: 68). In one embodiment when V2 is a scFv or a dsscFv, the linker connecting the variable domains VH and VL of V2 has the sequence GGGGSGGGGSGGGGSGGGGS (SEQ ID NO: 68). In one embodiment when V3 is a scFv or a dsscFv, the linker connecting the variable domains VH and VL of V3 has the sequence GGGGSGGGGSGGGGSGGGGS (SEQ ID NO: 68).

In one embodiment when V1 is a scFv or a dsscFv, the linker connecting the variable domains VH and VLof V1 has the sequence SGGGGSGGGGSGGGGS (SEQ ID NO: 69). In one embodiment when V2 is a scFv or a dsscFv, the linker connecting the variable domains VH and VL of V2 has the sequence SGGGGSGGGGSGGGGS (SEQ ID NO: 69). In one embodiment when V3 is a scFv or a dsscFv, the linker connecting the variable domains VH and VL of V3 has the sequence SGGGGSGGGGSGGGGS (SEQ ID NO: 69).

In one embodiment when V1 is a scFv or a dsscFv, the linker connecting the variable domains VH and VL of V1 has the sequence SGGGGSGGGGTGGGGS (SEQ ID NO: 70). In one embodiment when V2 is a scFv or a dsscFv, the linker connecting the variable domains VH and VL of V2 has the sequence SGGGGSGGGGTGGGGS SEQ ID NO: 70). In one embodiment when V3 is a scFv or a dsscFv, the linker connecting the variable domains VH and VL of V3 has the sequence SGGGGSGGGGTGGGGS SEQ ID NO: 70).

The present disclosure also provides sequences which are 80%, 90%, 91%, 92%, 93% 94%, 95% 96%, 97%, 98% or 99% similar to a sequence disclosed herein.

“Identity”, as used herein, indicates that at any particular position in the aligned sequences, the amino acid residue is identical between the sequences.

“Similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains).

Degrees of identity and similarity can be readily calculated (Computational Molecular Biology, Lesk, A.M., ed., Oxford University Press, New York, 1988; Biocomputing. Informatics and Genome Projects, Smith, D.W., ed., Academic Press, New York, 1993; Computer Analysis of Sequence Data, Part 1, Griffin, A.M., and Griffin, H.G., eds., Humana Press, New Jersey, 1994; Sequence Analysis in Molecular Biology, von Heinje, G., Academic Press, 1987, Sequence Analysis Primer, Gribskov, M. and Devereux, J., eds., M Stockton Press, New York, 1991, the BLAST™ software available from NCBI (Altschul, S.F. et al., 1990, J. Mol. Biol. 215:403-410; Gish, W. & States, D.J. 1993, Nature Genet. 3:266-272. Madden, T.L. et al., 1996, Meth. Enzymol. 266:131-141; Altschul, S.F. et al., 1997, Nucleic Acids Res. 25:3389-3402; Zhang, J. & Madden, T.L. 1997, Genome Res. 7:649-656,).

Multi-specific antibodies of the present invention may be generated by any suitable method known in the art.

Antibodies generated against an antigen polypeptide may be obtained, where immunisation of an animal is necessary, by administering the polypeptides to an animal, preferably a non-human animal, using well-known and routine protocols, see for example Handbook of Experimental Immunology, D. M. Weir (ed.), Vol 4, Blackwell Scientific Publishers, Oxford, England, 1986). Many warm-blooded animals, such as rabbits, mice, rats, sheep, cows, camels or pigs may be immunized. However, mice, rabbits, pigs and rats are generally most suitable.

Monoclonal antibodies may be prepared by any method known in the art such as the hybridoma technique (Kohler & Milstein, 1975, Nature, 256:495-497), the trioma technique, the human B-cell hybridoma technique (Kozbor et al 1983, Immunology Today, 4:72) and the EBV-hybridoma technique (Cole et al., Monoclonal Antibodies and Cancer Therapy, pp77-96, Alan R Liss, Inc., 1985).

Antibodies may also be generated using single lymphocyte antibody methods by cloning and expressing immunoglobulin variable region cDNAs generated from single lymphocytes selected for the production of specific antibodies by, for example, the methods described by Babcook, J. et al 1996, Proc. Natl. Acad. Sci. USA 93(15):7843-78481; WO92/02551; WO2004/051268 and WO2004/106377.

The antibodies for use in the present disclosure can also be generated using various phage display methods known in the art and include those disclosed by Brinkman et al. (in J. Immunol. Methods, 1995, 182: 41-50), Ames et al. (J. Immunol. Methods, 1995, 184:177-186), Kettleborough et al. (Eur. J. Immunol. 1994, 24:952-958), Persic et al. (Gene, 1997 187 9-18), Burton et al. (Advances in Immunology, 1994, 57:191-280) and WO90/02809; WO91/10737; WO92/01047; WO92/18619; WO93/11236; WO95/15982; WO95/20401; and US 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; 5,969,108, and WO20011/30305.

In one embodiment, the multi-specific antibodies according to the disclosure are humanised.

Humanised (which include CDR-grafted antibodies) as employed herein refers to molecules having one or more complementarity determining regions (CDRs) from a non-human species and a framework region from a human immunoglobulin molecule (see, e.g. US 5,585,089; WO91/09967). It will be appreciated that it may only be necessary to transfer the specificity determining residues of the CDRs rather than the entire CDR (see for example, Kashmiri et al., 2005, Methods, 36, 25-34). Humanised antibodies may optionally further comprise one or more framework residues derived from the non-human species from which the CDRs were derived.

As used herein, the term “humanised antibody” refers to an antibody wherein the heavy and/or light chain contains one or more CDRs (including, if desired, one or more modified CDRs) from a donor antibody (e.g. a murine monoclonal antibody) grafted into a heavy and/or light chain variable region framework of an acceptor antibody (e.g. a human antibody). For a review, see Vaughan et al, Nature Biotechnology, 16, 535-539, 1998. In one embodiment rather than the entire CDR being transferred, only one or more of the specificity determining residues from any one of the CDRs described herein above are transferred to the human antibody framework (see for example, Kashmiri et al., 2005, Methods, 36, 25-34). In one embodiment, only the specificity determining residues from one or more of the CDRs described herein above are transferred to the human antibody framework. In another embodiment, only the specificity determining residues from each of the CDRs described herein above are transferred to the human antibody framework.

When the CDRs or specificity determining residues are grafted, any appropriate acceptor variable region framework sequence may be used having regard to the class/type of the donor antibody from which the CDRs are derived, including mouse, primate and human framework regions. Suitably, the humanised antibody according to the present invention has a variable domain comprising human acceptor framework regions as well as one or more of the CDRs provided herein.

Examples of human frameworks which can be used in the present disclosure are KOL, NEWM, REI, EU, TUR, TEI, LAY and POM (Kabat et al supra). For example, KOL and NEWM can be used for the heavy chain, REI can be used for the light chain and EU, LAY and POM can be used for both the heavy chain and the light chain. Alternatively, human germline sequences may be used; these are available at: www2.mrc-lmb.cam.ac.uk/vbase/list2.php.

In a humanised antibody of the present disclosure, the acceptor heavy and light chains do not necessarily need to be derived from the same antibody and may, if desired, comprise composite chains having framework regions derived from different chains.

The framework regions need not have exactly the same sequence as those of the acceptor antibody. For instance, unusual residues may be changed to more frequently-occurring residues for that acceptor chain class or type. Alternatively, selected residues in the acceptor framework regions may be changed so that they correspond to the residue found at the same position in the donor antibody (see Reichmann et al 1998, Nature, 332, 323-324). Such changes should be kept to the minimum necessary to recover the affinity of the donor antibody. A protocol for selecting residues in the acceptor framework regions which may need to be changed is set forth in WO91/09967.

Derivatives of frameworks may have 1, 2, 3 or 4 amino acids replaced with an alternative amino acid, for example with a donor residue.

Donor residues are residues from the donor antibody, i.e. the antibody from which the CDRs were originally derived. Donor residues may be replaced by a suitable residue derived from a human receptor framework (acceptor residues).

In one embodiment the multi-specific antibodies of the present disclosure are fully human, in particular one or more of the variable domains are fully human.

Fully human antibodies are those in which the variable regions and the constant regions (where present) of both the heavy and the light chains are all of human origin, or substantially identical to sequences of human origin, not necessarily from the same antibody. Examples of fully human antibodies may include antibodies produced, for example by the phage display methods described above and antibodies produced by mice in which the murine immunoglobulin variable and optionally the constant region genes have been replaced by their human counterparts e.g. as described in general terms in EP0546073, US5,545,806, US5,569,825, US5,625,126, US5,633,425, US5,661,016, US5,770,429, EP 0438474 and EP0463151.

In one embodiment, the multi-specific antibodies of the disclosure are capable of selectively binding two, three or more different antigens of interest. In one embodiment, the multi-specific antibodies of the disclosure are capable of simultaneously binding two, three or more different antigens of interest.

In one embodiment, antigens of interest bound by the antigen binding domain formed by VH/VL, or V1 or V2 or V3 are independently selected from a cell-associated protein, for example a cell surface protein on cells such as bacterial cells, yeast cells, T-cells, B-cells, endothelial cells or tumour cells, and a soluble protein.

Antigens of interest may also be any medically relevant protein such as those proteins upregulated during disease or infection, for example receptors and/or their corresponding ligands. Particular examples of antigens include cell surface receptors such as T cell or B cell signalling receptors, co-stimulatory molecules, checkpoint inhibitors, natural killer cell receptors, Immunoglobulin receptors, TNFR family receptors, B7 family receptors, adhesion molecules, integrins, cytokine/chemokine receptors, GPCRs, growth factor receptors, kinase receptors, tissue-specific antigens, cancer antigens, pathogen recognition receptors, complement receptors, hormone receptors or soluble molecules such as cytokines, chemokines, leukotrienes, growth factors, hormones or enzymes or ion channels, epitopes, fragments and post translationally modified forms thereof.

In one embodiment, the multi-specific antibody of the disclosure may be used to functionally alter the activity of the antigen(s) of interest. For example, the antibody fusion protein may neutralize, antagonize or agonise the activity of said antigen, directly or indirectly.

In one embodiment, V1, V2 and V3 are specific for the same antigen, for example binding the same or a different epitope therein. In one embodiment, V3 is absent, and V1 and V2 are specific for the same antigens, for example the same or different epitopes on the same antigen. In one embodiment, V3 is absent, and V1 and V2 are specific for two different antigens.

In one embodiment, an antigen of interest bound by VH/VL or V1 or V2 or V3 provides the ability to recruit effector functions, such as complement pathway activation and/or effector cell recruitment.

The recruitment of effector function may be direct in that effector function is associated with a cell, said cell bearing a recruitment molecule on its surface. Indirect recruitment may occur when binding of an antigen to an antigen binding domain (such as V1 or V2 or V3) in the multi-specific antibody according to present disclosure to a recruitment polypeptide causes release of, for example, a factor which in turn may directly or indirectly recruit effector function, or may be via activation of a signalling pathway. Examples include IL2, IL6, IL8, IFN_{γ}, histamine, C1q, opsonin and other members of the classical and alternative complement activation cascades, such as C2, C4, C3-convertase, and C5 to C9.

As used herein, “a recruitment polypeptide” includes a FcyR such as FcyRI, FcyRII and FcyRIII, a complement pathway protein such as, but without limitation, C1q and C3, a CD marker protein (Cluster of Differentiation marker) or a fragment thereof which retains the ability to recruit cell-mediated effector function either directly or indirectly. A recruitment polypeptide also includes immunoglobulin molecules such as IgG1, IgG2, IgG3, IgG4, IgE and IgA which possess effector function.

In one embodiment, an antigen binding domain (such as V1 or V2 or V3 or VH/VL) in the multi-specific antibody according to the present disclosure has specificity for a complement pathway protein, with C1q being particularly preferred.

Further, multi-specific antibodies of the present disclosure may be used to chelate radionuclides by virtue of a single domain antibody which binds to a nuclide chelator protein. Such fusion proteins are of use in imaging or radionuclide targeting approaches to therapy.

In one embodiment an antigen binding domain within a multi-specific antibody according to the disclosure (such as V1 or V2 or V3 or VH/VL) has specificity for a serum carrier protein, a circulating immunoglobulin molecule, or CD35/CR1, for example for providing an extended half-life to the antibody fragment with specificity for said antigen of interest by binding to said serum carrier protein, circulating immunoglobulin molecule or CD35/CR1.

As used herein, “serum carrier proteins” include thyroxine-binding protein, transthyretin, α1-acid glycoprotein, transferrin, fibrinogen and albumin, or a fragment of any thereof.

As used herein, a “circulating immunoglobulin molecule” includes IgG1, IgG2, IgG3, IgG4, sIgA, IgM and IgD, or a fragment of any thereof.

CD35/CR1 is a protein present on red blood cells which have a half-life of 36 days (normal range of 28 to 47 days; Lanaro et al., 1971, Cancer, 28(3):658-661).

In one embodiment, the antigen of interest for which VH/VL has specificity is a serum carrier protein, such as a human serum carrier, such as human serum albumin.

In one embodiment, the antigen of interest for which V1 has specificity is a serum carrier protein, such as a human serum carrier, such as human serum albumin. Thus, in one embodiment, V1 comprises an albumin binding domain.

In one embodiment, the antigen of interest for which V2 has specificity is a serum carrier protein, such as a human serum carrier, such as human serum albumin. Thus, in one embodiment, V2 comprises an albumin binding domain.

In one embodiment, the antigen of interest for which V3 has specificity is a serum carrier protein, such as a human serum carrier, such as human serum albumin. Thus, in one embodiment, V3 comprises an albumin binding domain.

In one embodiment only one of VH/VL_{,} V1 ₒᵣ V2 ₒᵣ V3 has specificity for a serum carrier protein, such as a human serum carrier, such as human serum albumin. Thus, in one embodiment only one of VH/VL_{,} V1 ₒᵣ V2 ₒᵣ V3 comprises an albumin binding domain.

In one embodiment, the albumin binding domain further binds protein A. In one embodiment, the albumin binding domain comprises 6 CDRs, for example SEQ ID NO: 71 for CDRH1, SEQ ID NO: 72 for CDRH2, SEQ ID NO: 73 for CDRH3, SEQ ID NO: 74 for CDRL1, SEQ ID NO: 75 for CDRL2 and SEQ ID NO: 76 for CDRL3. In one embodiment, the said 6 CDRs SEQ ID NO: 71 to 76 are in the position VH/VL in the constructs of the present disclosure. In one embodiment the said 6 CDRs SEQ ID NO: 71 to 76 are in the position V1 in the constructs of the present disclosure. In one embodiment the said 6 CDRs SEQ ID NO: 71 to 76 are in the position VH/VL and V1 in the constructs of the present disclosure.

In one embodiment, the albumin binding domain comprises a heavy chain variable domain selected from SEQ ID NO: 77 and SEQ ID NO: 78 and a light chain variable domain selected from SEQ ID NO: 79 and SEQ ID NO: 80, in particular SEQ ID NO: 77 and 79 or SEQ ID NO: 78 and 80 for the heavy and light chain respectively. In one embodiment, the albumin binding domain is a scFv of sequence SEQ ID NO: 81. In one embodiment, the albumin binding domain is a dsscFv of sequence SEQ ID NO: 82, as shown below:

645 scFv (VH/VL) (SEQ ID NO: 81):

EVQLLESGGGLVQPGGSLRLSCAVSGIDLSNYAINWVRQAPGKGLEWIGI
IWASGTTFYATWAKGRFTISRDNSKNTVYLQMNSLRAEDTAVYYCARTVP
GYSTAPYFDLWGQGTLVTVSSGGGGSGGGGSGGGGSGGGGSDIQMTQSPS
SVSASVGDRVTITCQSSPSVWSNFLSWYQQKPGKAPKLLIYEASKLTSGV
PSRFSGSGSGTDFTLTISSLQPEDFATYYCGGGYSSISDTTFGGGTKVEI
K

645 dsscFv (VH/VL) (with cysteines engineered for a disulphide bond, underlined) (SEQ ID NO: 82):

EVQLLESGGGLVQPGGSLRLSCAVSGIDLSNYAINWVRQAPGKCLEWIGI
IWASGTTFYATWAKGRFTISRDNSKNTVYLQMNSLRAEDTAVYYCARTVP
GYSTAPYFDLWGQGTLVTVSSGGGGSGGGGSGGGGSGGGGSDIQMTQSPS
SVSASVGDRVTITCQSSPSVWSNFLSWYQQKPGKAPKLLIYEASKLTSGV
PSRFSGSGSGTDFTLTISSLQPEDFATYYCGGGYSSISDTTFGCGTKVEI
K

In one embodiment, these domains are in the position VH/VL in the constructs of the present disclosure. In one embodiment, these variable domains are in the position V1. In one embodiment, these variable domains are in the position VH/VL and V1 in the constructs of the present disclosure. When the variable domains are in two locations in the constructs of the present disclosure, the same pair of variable domains may be in each location or two different pairs of variable domains may be employed.

In one embodiment the multi-specific antibodies of the present disclosure are processed to provide improved affinity for a target antigen or antigens. Such variants can be obtained by a number of affinity maturation protocols including mutating the CDRs (Yang et al J. Mol. Biol., 254, 392-403, 1995), chain shuffling (Marks et al., Bio/Technology, 10, 779-783, 1992), use of mutator strains of E. coli (Low et al., J. Mol. Biol., 250, 359-368, 1996), DNA shuffling (Patten et al., Curr. Opin. Biotechnol., 8, 724-733, 1997), phage display (Thompson et al., J. Mol. Biol., 256, 77-88, 1996) and sexual PCR (Crameri et al Nature, 391, 288-291, 1998). Vaughan et al (supra) discusses these methods of affinity maturation.

Improved affinity as employed herein in this context refers to an improvement over the starting molecule.

If desired a multi-specific antibody construct for use in the present disclosure may be conjugated to one or more effector molecule(s). It will be appreciated that the effector molecule may comprise a single effector molecule or two or more such molecules so linked as to form a single moiety that can be attached to the antibodies of the present invention. Where it is desired to obtain an antibody fragment linked to an effector molecule, this may be prepared by standard chemical or recombinant DNA procedures in which the antibody fragment is linked either directly or via a coupling agent to the effector molecule. Techniques for conjugating such effector molecules to antibodies are well known in the art (see, Hellstrom et al Controlled Drug Delivery, 2nd Ed., Robinson et al eds., 1987, pp. 623-53; Thorpe et al 1982, Immunol. Rev., 62:119-58 and Dubowchik et al 1999, Pharmacology and Therapeutics, 83, 67-123). Particular chemical procedures include, for example, those described in WO93/06231, WO92/22583, WO89/00195, WO89/01476 and WO03031581. Alternatively, where the effector molecule is a protein or polypeptide the linkage may be achieved using recombinant DNA procedures, for example as described in WO86/01533 and EP0392745.

The term “effector molecule” as used herein includes, for example, biologically active proteins, for example enzymes, other antibody or antibody fragments, synthetic or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

Other effector molecules may include chelated radionuclides such as 111In and 90Y, Lu177, Bismuth213, Californium252, Iridium192 and Tungsten188/Rhenium188; or drugs such as but not limited to, alkylphosphocholines, topoisomerase I inhibitors, taxoids and suramin.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions.

In another embodiment the effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO05/117984.

Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero- polysaccharide.

Specific optional substituents which may be present on the above-mentioned synthetic polymers include one or more hydroxy, methyl or methoxy groups.

“Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

The size of the polymer may be varied as desired, but will generally be in an average molecular weight range from 500 Da to 50000 Da, for example from 5000 to 40000 Da such as from 20000 to 40000 Da. The polymer size may in particular be selected on the basis of the intended use of the product for example ability to localize to certain tissues such as tumors or extend circulating half-life (for review see Chapman, 2002, Advanced Drug Delivery Reviews, 54, 531-545).

Suitable polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

In one embodiment antibodies for use in the present disclosure are attached to poly(ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example US5,219,996; US 5,667,425; WO98/25971, WO2008/038024). In one embodiment the antibody molecule of the present invention comprises a modified Fab fragment wherein the modification is the addition to the C-terminal end of its heavy chain one or more amino acids to allow the attachment of an effector molecule. Suitably, the additional amino acids form a modified hinge region containing one or more cysteine residues to which the effector molecule may be attached. Multiple sites can be used to attach two or more PEG molecules.

Suitably PEG molecules are covalently linked through a thiol group of at least one cysteine residue located in the antibody fragment. Each polymer molecule attached to the modified antibody fragment may be covalently linked to the sulphur atom of a cysteine residue located in the fragment. The covalent linkage will generally be a disulphide bond or, in particular, a sulphur-carbon bond. Where a thiol group is used as the point of attachment appropriately activated effector molecules, for example thiol selective derivatives such as maleimides and cysteine derivatives may be used. An activated polymer may be used as the starting material in the preparation of polymer-modified antibody fragments as described above. The activated polymer may be any polymer containing a thiol reactive group such as an α-halocarboxylic acid or ester, e.g. iodoacetamide, an imide, e.g. maleimide, a vinyl sulphone or a disulphide. Such starting materials may be obtained commercially (for example from Nektar, formerly Shearwater Polymers Inc., Huntsville, AL, USA) or may be prepared from commercially available starting materials using conventional chemical procedures. Particular PEG molecules include 20K methoxy-PEG-amine (obtainable from Nektar, formerly Shearwater; Rapp Polymere; and SunBio) and M-PEG-SPA (obtainable from Nektar, formerly Shearwater).

In one embodiment, a F(ab′)₂, Fab or Fab′ in the molecule is PEGylated, i.e. has PEG (poly(ethyleneglycol)) covalently attached thereto, e.g. according to the method disclosed in EP 0948544 or EP1090037 [see also “Poly(ethyleneglycol) Chemistry, Biotechnical and Biomedical Applications”, 1992, J. Milton Harris (ed), Plenum Press, New York, “Poly(ethyleneglycol) Chemistry and Biological Applications”, 1997, J. Milton Harris and S. Zalipsky (eds), American Chemical Society, Washington DC and “Bioconjugation Protein Coupling Techniques for the Biomedical Sciences”, 1998, M. Aslam and A. Dent, Grove Publishers, New York; Chapman, A. 2002, Advanced Drug Delivery Reviews 2002, 54:531-545]. In one embodiment PEG is attached to a cysteine in the hinge region. In one example, a PEG modified Fab fragment has a maleimide group covalently linked to a single thiol group in a modified hinge region. A lysine residue may be covalently linked to the maleimide group and to each of the amine groups on the lysine residue may be attached a methoxypoly(ethyleneglycol) polymer having a molecular weight of approximately 20,000 Da. The total molecular weight of the PEG attached to the Fab fragment may therefore be approximately 40,000 Da.

Particular PEG molecules include 2-[3-(N-maleimido)propionamido]ethyl amide of N,N′-bis(methoxypoly(ethylene glycol) MW 20,000) modified lysine, also known as PEG2MAL40K (obtainable from Nektar, formerly Shearwater).

Alternative sources of PEG linkers include NOF who supply GL2-400MA2 (wherein m in the structure below is 5) and GL2-400MA (where m is 2) and n is approximately 450:

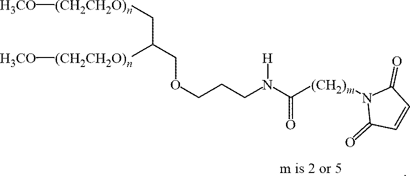

That is to say each PEG is about 20,000 Da.

Further alternative PEG effector molecules of the following type:

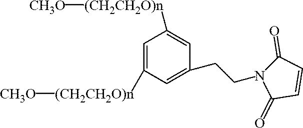

are available from Dr Reddy, NOF and Jenkem.

In one embodiment, there is provided an antibody molecule which is PEGylated (for example with a PEG described herein), attached through a cysteine amino acid residue at or about amino acid 226 in the chain, for example amino acid 226 of the heavy chain (by sequential numbering).

In one embodiment there is provided a polynucleotide sequence encoding a multi-specific antibody of the present disclosure, such as a DNA sequence.

In one embodiment there is provided a polynucleotide sequence encoding one or more, such as two or more, or three or more polypeptide components of a multi-specific antibody of the present disclosure, for example:
a polypeptide chain of

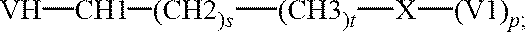

and
a polypeptide chain of

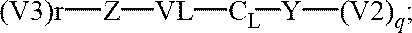

wherein:

| | |
|---|---|
| VH | represents a heavy chain variable domain; |
| CH1 | represents domain 1 of a heavy chain constant region; |
| CH2 | represents domain 2 of a heavy chain constant region; |
| CH3 | represents domain 3 of a heavy chain constant region; |
| X | represents a bond or linker; |
| V1 | represents a dsscFv, a dsFv, a scFv, a VH, a VL or a VHH; |
| V3 | represents a dsscFv, a dsFv, a scFv, a VH, a VL or a VHH; |
| Z | represents a bond or linker; |
| VL | represents a light chain variable domain; |
| C_{L} | represents a domain from a light chain constant region, such as Ckappa; |
| Y | represents a bond or linker; |
| V2 | represents a dsscFv, a dsFv, a scFv, a VH, a VL or a VHH; |
| p | represents 0 or 1; |
| q | represents 0 or 1; |
| r | represents 0 or 1; |
| s | represents 0 or 1; |
| t | represents 0 or 1; |

wherein when p is 0, X is absent and when q is 0, Y is absent and when r is 0, Z is absent; and
wherein when q is 0, r is 1 and when r is 0, q is 1; and
wherein the polypeptide chain of formula (II) comprises at least one dsscFv, dsFv, scFv, VH or VHH; and
wherein the polypeptide chain of formula (I) comprises a protein A binding domain; and
wherein the polypeptide chain of formula (II) does not bind protein A.

In one embodiment, the polynucleotide, such as the DNA is comprised in a vector.

The skilled person will appreciate that when V1 and/or V2 and/or V3 represents a dsFv, the multi-specific antibody will comprise a third polypeptide encoding the corresponding free VH or VL domain which is not attached to X or Y or Z. Accordingly, the multi-specific antibody of the present invention may be encoded by one or more, two or more or three or more polynucleotides and these may be incorporated into one or more vectors.

General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

Also provided is a host cell comprising one or more cloning or expression vectors comprising one or more DNA sequences encoding a multi-specific protein of the present invention. Any suitable host cell/vector system may be used for expression of the DNA sequences encoding the antibody of the present invention. Bacterial, for example E. coli, and other microbial systems may be used or eukaryotic, for example mammalian, host cell expression systems may also be used. Suitable mammalian host cells include HEK, e.g. HEK293, CHO, myeloma, NSO myeloma cells and SP2 cells, COS cells or hybridoma cells.

The present disclosure also provides a process for the production of a multi-specific antibody according to the present disclosure comprising culturing a host cell containing a vector of the present invention under conditions suitable for leading to expression of protein from DNA encoding the multi-specific antibody of the present invention, and isolating the multi-specific antibody.

For production of products comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides. In one example, the cell line may be transfected with two vectors, each encoding a polypeptide chain of an antibody of the present invention. Where V1 and/or V2 and/or V3 are a dsFv, the cell line may be transfected with three vectors, each encoding a polypeptide chain of a multi-specific antibody of the invention.

In one embodiment, the cell line is transfected with two vectors each one encoding a different polypeptide selected from:
a polypeptide chain of formula (I):

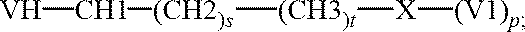

and
a polypeptide chain of formula (II):

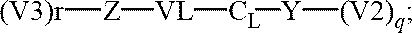

wherein:

| | |
|---|---|
| VH | represents a heavy chain variable domain; |
| CH1 | represents domain 1 of a heavy chain constant region; |
| CH2 | represents domain 2 of a heavy chain constant region; |
| CH3 | represents domain 3 of a heavy chain constant region; |
| X | represents a bond or linker; |
| V1 | represents a dsscFv, a dsFv, a scFv, a VH, a VL or a VHH; |
| V3 | represents a dsscFv, a dsFv, a scFv, a VH, a VL or a VHH; |
| Z | represents a bond or linker; |
| VL | represents a light chain variable domain; |
| C_{L} | represents a domain from a light chain constant region, such as Ckappa; |
| Y | represents a bond or linker; |
| V2 | represents a dsscFv, a dsFv, a scFv, a VH, a VL or a VHH; |
| p | represents 0 or 1; |
| q | represents 0 or 1; |
| r | represents 0 or 1; |
| s | represents 0 or 1; |
| t | represents 0 or 1; |

wherein when p is 0, X is absent and when q is 0, Y is absent and when r is 0, Z is absent; and
wherein when q is 0, r is 1 and when r is 0, q is 1; and
wherein the polypeptide chain of formula (II) comprises at least one dsscFv, dsFv, scFv, VH or VHH; and
wherein the polypeptide chain of formula (I) comprises a protein A binding domain; and
wherein the polypeptide chain of formula (II) does not bind protein A.

In one embodiment when V1 is a dsFv and the VH domain of V1 is attached to X, the cell line may be transfected with a third vector which encodes the VL domain of V1.

In one embodiment when V1 is a dsFv and the VL domain of V1 is attached to X, the cell line may be transfected with a third vector which encodes the VH domain of V1.

In one embodiment when V2 is a dsFv and the VH domain of V2 is attached to Y, the cell line may be transfected with a third vector which encodes the VL domain of V2.

In one embodiment when V2 is a dsFv and the VL domain of V2 is attached to Y, the cell line may be transfected with a third vector which encodes the VH domain of V2.

In one embodiment when V3 is a dsFv and the VH domain of V3 is attached to Y, the cell line may be transfected with a third vector which encodes the VL domain of V3.

In one embodiment when V3 is a dsFv and the VL domain of V3 is attached to Y, the cell line may be transfected with a third vector which encodes the VH domain of V3.

In one embodiment when V3 is absent and when both V1 and V2 are a dsFv and the VL domain of V2 is attached to Y and the VL domain of V1 is attached to X, the cell line may be transfected with a third vector which encodes the common VH domain of both V1 and V2.

In one embodiment when V3 is absent and when both V1 and V2 are a dsFv and the VH domain of V2 is attached to Y and the VH domain of V1 is attached to X, the cell line may be transfected with a third vector which encodes the common VL domain of both V1 and V2.

It will be appreciated that the ratio of each vector transfected into the host cell may be varied in order to optimise expression of the multi-specific antibody product. In one embodiment where two vectors are used, one coding the polypeptide chain of formula (I) i-e the heavy chain, and another one coding the polypeptide chain of formula (II), i-e the light chain, the ratio of vectors (LC containing vector): (HC containing vector) may be comprised between 1:1, 5:1, preferably between 1,5:1 and 5:1, e.g. the ratio may be 2:1, 3:1, 4:1, 5:1. In one embodiment where three vectors are used, the ratio of vectors (LC containing vector): (HC containing vector): free domain containing vector may be comprised between 1:1:1 and 5:1:1. It will be appreciated that the skilled person is able to find an optimal ratio by routine testing of protein expression levels following transfection. Alternatively, or in addition, the levels of expression of each polypeptide chain of the multi-specific construct from each vector may be controlled by using the same or different promoters.

It will be appreciated that two or more or where present, three of the polypeptide components may be encoded by a polynucleotide in a single vector. It will also be appreciated that where two or more, in particular three or more, of the polypeptide components are encoded by a polynucleotide in a single vector the relative expression of each polypeptide component can be varied by utilising different promoters for each polynucleotide encoding a polypeptide component of the present disclosure.

In one embodiment, the vector comprises a single polynucleotide sequence encoding two or where present, three, polypeptide chains of the multi-specific antibody of the present invention under the control of a single promoter.

In one embodiment, the vector comprises a single polynucleotide sequence encoding two, or where present, three, polypeptide chains of the multi-specific antibody of the present disclosure wherein each polynucleotide sequence encoding each polypeptide chain is under the control of a different promoter.

In one aspect, the invention provides a method for producing a multi-specific antibody comprising a polypeptide chain of formula (I) and a polypeptide chain of formula (II) as defined above, said method comprising:
a) Expressing a polypeptide chain of formula (I) and a polypeptide chain of formula (II) as defined above, in a host cell, wherein the polypeptide chain of formula (II) is in excess over the polypeptide chain of formula (I); and
b) Recovering the composition of polypeptides expressed at step a), said composition comprising a multi-specific antibody and a LC dimer of formula (II-II); and
c) Purifying the multi-specific antibody, wherein when s is 1 and t is 1, said multi-specific antibody is purified as a dimer with two heavy chains of formula (I) and two associated light chains of formula (II) and, wherein when s is 0 and t is 0, said multi-specific antibody is purified as a dimer with one heavy chain of formula (I) and one associated light chain of formula (II); and,
   wherein the polypeptide chain of formula (II) comprises at least one dsscFv, dsFv, scFv, VH or VHH; and,
   wherein the polypeptide chain of formula (I) comprises a protein A binding domain; and,
   wherein the polypeptide chain of formula (II) does not bind protein A; and,
   wherein step c) comprises subjecting the composition of polypeptides recovered at step b), optionally following at least one purification step, to a Protein A affinity chromatography column.

Means for expressing the light chain in excess over the heavy chain are well known in the art and include for example varying the ratio of vectors used for the transfection of a host cell as described above. In one embodiment, two vectors are used, one coding the polypeptide chain of formula (I) i-e the heavy chain, and another one coding the polypeptide chain of formula (II), i-e the light chain, wherein the ratio of vectors (LC containing vector): (HC containing vector) is comprised between 1,5:1 and 5:1, for example is 1,5:1, 2:1, 3:1, 4:1, 5:1. In another embodiment, a unique expression vector is used, comprising transcription units coding the LC in excess over the transcription units coding the HC. In another embodiment, the same quantity of vector or transcription units is used, but said vector or transcription units comprise a modified transcription or translation regulatory element (e.g. a promoter) in the LC coding unit which is absent from the HC coding unit and promotes the over-expression of the LC.

In one embodiment, step c) comprises a clarification step. Means for clarification are well known in the art and include centrifugation, filtration, flocculation, and pH adjustments, in order to remove impurities including cell components and other debris. In one embodiment, step c) comprises subjecting the composition of polypeptides recovered at step b), following a clarification step, to a Protein A affinity chromatography column. In such embodiment, the composition of polypeptides recovered at step b) is first clarified, then loaded onto a Protein A affinity chromatography column. In another embodiment, step c) comprises only one purification step, i-e the protein A purification step.

In one embodiment, the method for producing a multi-specific antibody of the invention does not comprise a protein L affinity chromatography.

Advantageously, the inventors have re-engineered the multi-specific antibodies disclosed in the prior art to provide improved multi-specific antibodies that can be easily and efficiently purified using a protein A purification step, without requiring any additional purification step. The polypeptide of formula (II) of the antibody of the present invention does not bind protein A, such that only the multi-specific antibody binds to protein A, via its heavy chain, and the LC dimers are maintained in the unbound fraction.

In one embodiment, less than 5%, preferably less than 4%, or less than 3%, or less than 2%, and more preferably less than 1 % of the LC dimer of formula (II-II) is co-purified with the multi-specific antibody, said multi-specific antibody being purified as a dimer with two heavy chains of formula (I) and two associated light chains of formula (II) when s is 1 and t is 1 and, as a dimer with one heavy chain of formula (I) and one associated light chain of formula (II) when s is 0 and t is 0.

In another aspect, there is provided a process for purifying a multi-specific antibody comprising a polypeptide chain of formula (I) and a polypeptide chain of formula (II) as defined above, said method comprising:
a) Obtaining a composition of polypeptide chains of formula (I) and polypeptide chains of formula (II) as defined above, said composition comprising a multi-specific antibody,
   wherein when s is 1 and t is 1, the multi-specific antibody is a dimer with two heavy chains of formula (I) and two associated light chains of formula (II) and; when s is 0 and t is 0, the multi-specific antibody is a dimer with one heavy chain of formula (I) and one associated light chain of formula (II); and a dimer of two light chains of formula (II-II), associated together (LC dimer); and,
   wherein the polypeptide chain of formula (II) comprises at least one dsscFv, dsFv, scFv, VH or VHH; and,
   wherein the polypeptide chain of formula (I) comprises a protein A binding domain; and,
   wherein the polypeptide chain of formula (II) does not bind protein A; and
b) Loading the composition obtained in step a), onto a protein A affinity column, such that the multi-specific antibody is retained on the column whilst the LC dimer does not bind to the column; and
c) Washing the protein A affinity column; and,
d) Eluting the multi-specific antibody; and,
e) Recovering the multi-specific antibody.

In one embodiment, the composition loaded onto the protein A column has been clarified. Several protein A columns can be used, in particular native protein A columns, for example a column MabSelect (GE Healthcare). In one embodiment, the protein A affinity column is a MabSelect column. In one embodiment, the protein A is a variant of a naturally occurring protein A, said protein A variant maintaining its ability to bind VH3 domains. The loading (or binding) step may be performed at pH 7-8, for example 7.4. The composition obtained in step a) may be loaded onto the protein A affinity column during a 5, 10 or 15 minutes contact time. In one embodiment, the loading step b) is performed with a binding buffer comprising 200 mM glycine, pH7.5.

In one embodiment, the elution step d) is performed under acidic conditions. In one embodiment, the elution step d) is performed at a pH comprised between 2 and 4.5, preferably at a pH comprised between 3 and 4. In one embodiment, step d) is a 0.1 M sodium citrate pH3.1 elution step. In one embodiment, step d) is a 0.1 M sodium citrate pH3.2 elution step. In one embodiment, step d comprises a first elution step with 0.1 M sodium citrate pH3.2, and a second elution step with 0.1 M Citrate pH2.1. Alternatively, the elution at step d) may be performed under chaotropic conditions or any other condition promoting the elution of the bound multi-specific antibody, including gentle elution.

In one embodiment, the process for purifying a multi-specific antibody comprises at least one additional purification step, before or after step d).

For example, the process may further comprise of additional chromatography step(s) to ensure product and process related impurities are appropriately resolved from the product stream, including ion (cation or anion) exchange chromatography, hydrophobic interaction chromatography, and mixed mode chromatography. The purification process may also comprise of one or more ultra-filtration steps, such as a concentration and diafiltration step.

Purified form as used supra is intended to refer to at least 90% purity, such as 91, 92, 93, 94, 95, 96, 97, 98, 99% w/w or more pure.

Substantially free of endotoxin is generally intended to refer to an endotoxin content of 1 EU per mg antibody product or less such as 0.5 or 0.1 EU per mg product.

Substantially free of host cell protein or DNA is generally intended to refer to host cell protein and/or DNA content 400 µg per mg of antibody product or less such as 100 µg per mg or less, in particular 20 µg per mg, as appropriate.

The multi-specific proteins according to the present disclosure are expressed at good levels from host cells. Thus, the properties of the antibodies and/or fragments appear to be optimised and conducive to commercial processing.

Advantageously, the multi-specific antibodies of the present disclosure minimise the amount of aggregation seen after purification and maximise the amount of monomer in the formulations of the construct at pharmaceutical concentrations, for example the monomer may be present as 50%, 60%, 70% or 75% or more, such as 80 or 90% or more such as 91, 92, 93, 94, 95, 96, 97, 98 or 99% or more of the total protein. In one example, a purified sample of a multi-specific antibody of the present disclosure remains greater than 98% or 99% monomeric after 28 days storage at 4° C. In one example, a purified sample of a multi-specific antibody of the present disclosure at 5 mg/ml in phosphate buffered saline (PBS) remains greater than 98% monomeric after 28 days storage at 4° C. Monomer yield may be determined using any suitable method, such as size exclusion chromatography.

The antibodies of the present disclosure and compositions comprising the same are useful in the treatment, for example in the treatment and/or prophylaxis of a pathological condition.

The present disclosure also provides a pharmaceutical or diagnostic composition comprising an antibody of the present disclosure in combination with one or more of a pharmaceutically acceptable excipient, diluent or carrier. Accordingly, provided is the use of an antibody of the present disclosure for use in treatment and for the manufacture of a medicament, in particular for an indication disclosed herein.

The composition will usually be supplied as part of a sterile, pharmaceutical composition that will normally include a pharmaceutically acceptable carrier. A pharmaceutical composition of the present disclosure may additionally comprise a pharmaceutically-acceptable adjuvant.

The present disclosure also provides a process for preparation of a pharmaceutical or diagnostic composition comprising adding and mixing the antibody of the present disclosure together with one or more of a pharmaceutically acceptable excipient, diluent or carrier.

The antibody may be the sole active ingredient in the pharmaceutical or diagnostic composition or may be accompanied by other active ingredients.

In a further embodiment the antibody, fragment or composition according to the disclosure is employed in combination with a further pharmaceutically active agent.

The pharmaceutical compositions suitably comprise a therapeutically effective amount of the antibody of the invention. The term “therapeutically effective amount” as used herein refers to an amount of a therapeutic agent needed to treat, ameliorate or prevent a targeted disease or condition, or to exhibit a detectable therapeutic or preventative effect. For any antibody, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. This amount can be determined by routine experimentation and is within the judgement of the clinician.

Compositions may be administered individually to a patient or may be administered in combination (e.g. simultaneously, sequentially or separately) with other agents, drugs or hormones.

The dose at which the antibody of the present disclosure is administered depends on the nature of the condition to be treated, the extent of the inflammation present and on whether the antibody is being used prophylactically or to treat an existing condition.

The frequency of dose will depend on the half-life of the antibody and the duration of its effect.

The pharmaceutically acceptable carrier should not itself induce the production of antibodies harmful to the individual receiving the composition and should not be toxic. Pharmaceutically acceptable carriers are well known in the art.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the patient.

Suitable forms for administration include forms suitable for parenteral administration, e.g. by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilising and/or dispersing agents. Alternatively, the antibody may be in dry form, for reconstitution before use with an appropriate sterile liquid.

Once formulated, the compositions of the invention can be administered directly to the subject. The subjects to be treated can be animals. However, in one or more embodiments the compositions are adapted for administration to human subjects.

The pharmaceutical compositions of this disclosure may be administered by any number of routes including, but not limited to, oral, intravenous, intramuscular, intra-arterial, intramedullary, intrathecal, intraventricular, transdermal, transcutaneous, subcutaneous, intraperitoneal, intranasal, enteral, topical, sublingual, intravaginal or rectal routes. Typically, the therapeutic compositions may be prepared as injectables, either as liquid solutions or suspensions. Solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a specific tissue of interest. Dosage treatment may be a single dose schedule or a multiple dose schedule.

It will be appreciated that the active ingredient in the composition will be an antibody. As such, it will be susceptible to degradation in the gastrointestinal tract. Thus, if the composition is to be administered by a route using the gastrointestinal tract, the composition will advantageously contain agents which protect the antibody from degradation but which release the antibody once it has been absorbed from the gastrointestinal tract.

The pathological condition or disorder, may, for example be selected from the group consisting of infections (viral, bacterial, fungal and parasitic), endotoxic shock associated with infection, arthritis such as rheumatoid arthritis, asthma such as severe asthma, chronic obstructive pulmonary disease (COPD), pelvic inflammatory disease, Alzheimer’s Disease, inflammatory bowel disease, Crohn’s disease, ulcerative colitis, Peyronie’s Disease, coeliac disease, gallbladder disease, Pilonidal disease, peritonitis, psoriasis, vasculitis, surgical adhesions, stroke, Type I Diabetes, lyme disease, meningoencephalitis, autoimmune uveitis, immune mediated inflammatory disorders of the central and peripheral nervous system such as multiple sclerosis, lupus (such as systemic lupus erythematosus) and Guillain-Barr syndrome, Atopic dermatitis, autoimmune hepatitis, fibrosing alveolitis, Grave’s disease, IgA nephropathy, idiopathic thrombocytopenic purpura, Meniere’s disease, pemphigus, primary biliary cirrhosis, sarcoidosis, scleroderma, Wegener’s granulomatosis, other autoimmune disorders, pancreatitis, trauma (surgery), graft-versus-host disease, transplant rejection, heart disease including ischaemic diseases such as myocardial infarction as well as atherosclerosis, intravascular coagulation, bone resorption, osteoporosis, osteoarthritis, periodontitis and hypochlorhydia.

The present disclosure also provides a multi-specific antibody according to the present invention for use in the treatment or prophylaxis of pain, particularly pain associated with inflammation.

Thus, there is provided a multi-specific antibody according to the present disclosure for use in treatment and methods of treatment employing same.

The quantity of an antibody of the invention required for the prophylaxis or treatment of a particular condition will vary depending on the antibody and the condition to be treated.

The antibody of the present invention may also be used in diagnosis, for example in the in vivo diagnosis and imaging of disease states.

“Comprising” in the context of the present specification is intended to meaning including. Where technically appropriate, embodiments of the invention may be combined. Embodiments are described herein as comprising certain features/elements. The disclosure also extends to separate embodiments consisting or consisting essentially of said features/elements.

Technical references such as patents and applications are incorporated herein by reference. Any embodiments specifically and explicitly recited herein may form the basis of a disclaimer either alone or in combination with one or more further embodiments.

The present disclosure is further described by way of illustration only in the following examples, which refer to the accompanying Figures, in which:

### BRIEF DESCRIPTION OF THE FIGURES

FIG. 1: Sequences of anti-albumin 645 antibody

FIG. 2: Analysis of final purified TrYbe 03. FIG. 2A: BEH200 SEC-UPLC (vertical axis; EU (Emission Unit), horizontal axis; time (in minutes)). FIG. 2B: SDS-PAGE (lane M:Mark12™; lane 1: non-reducing conditions; lane 2: reducing conditions).

FIG. 3: Schematics of Wittrup (Wittrup 01 and Wittrup 02) and TrYbe antibodies (TrYbe 03 to TrYbe 06) and corresponding LC dimers. All Wittrup molecules have a common hglFL and Fab region. All TrYbe molecules have a common Fab region.

FIG. 4: Reducing (FIG. 4A) and Non-Reducing (FIG. 4B) SDS-PAGE analysis of Protein A and Protein L chromatography including Load materials, Eluates and Flow throughs for Wittrup 01 and Wittrup 02 molecules. Samples loaded as follows: Lane M: Mark12™; Lanes 1A-1E: Wittrup 01 (1A: Protein A Load (Supernatant); 1B: Protein A Eluate; 1C: Protein L Load (Protein A flow through); 1D: Protein L Eluate; 1E: Protein L flow through); Lanes 2A-2E: Wittrup 02 (2A: Protein A Load (Supernatant); 2B: Protein A Eluate; 2C: Protein L Load (Protein A flow through); 2D: Protein L Eluate; 2E: Protein L flow through).

FIG. 5: Reducing (FIG. 5A) and Non-Reducing (FIG. 5B) SDS-PAGE analysis of Protein A and Protein L chromatography including Load materials, Eluates and Flow throughs for TrYbe 03 and TrYbe 04 molecules. Samples loaded as follows: Lane M: Mark12™; Lanes 3A-3E: TrYbe 03 (3A: Protein A Load (Supernatant); 3B: Protein A Eluate; 3C: Protein L Load (Protein A flow through); 3 D: Protein L Eluate; 3E: Protein L flow through); Lanes 4A-4E: TrYbe 04 (4A: Protein A Load (Supernatant); 4B: Protein A Eluate; 4C: Protein L Load (Protein A flow through); 4D: Protein L Eluate; 4E: Protein L flow through). FIG. 5C: Densitometrical analysis of reducing SDS-PAGE. Samples include Protein A Eluates of TrYbe 03 and TrYbe 04 (horizontal axis). Analysis is displayed as a percentage relative to the density of the heavy chain band in the vertical axis.

FIG. 6: Reducing (FIG. 6A) and Non-Reducing (FIG. 6B) SDS-PAGE analysis of Protein A and Protein L chromatography including Load materials, Eluates and Flow throughs for TrYbe 03 and TrYbe 05 molecules. Samples loaded as follows: Lane M: Mark12™; Lanes 3A-3E: TrYbe 03 (3A: Protein A Load (Supernatant); 3B: Protein A Eluate; 3C: Protein L Load (Protein A flow through); 3D: Protein L Eluate; 3E: Protein L flow through); Lanes 5A-5E: TrYbe 05 (5A: Protein A Load (Supernatant); 5B: Protein A Eluate; 5C: Protein L Load (Protein A flow through); 5D: Protein L Eluate; 5E: Protein L flow through). FIG. 6C: Densitometrical analysis of reducing SDS-PAGE. Samples include Protein A Eluates of TrYbe 03 and TrYbe 05 (horizontal axis). Analysis is displayed as a percentage relative to the density of the heavy chain band in the vertical axis.

FIG. 7: Reducing (FIG. 7A) and Non-Reducing (FIG. 7B) SDS-PAGE analysis of Protein A and Protein L chromatography including Load materials, Eluates and Flow throughs for TrYbe 04 and TrYbe 06 molecules. Samples loaded as follows: Lane M: Mark12™; Lanes 4A-4E: TrYbe 04 (4A: Protein A Load (Supernatant); 4B: Protein A Eluate; 4C: Protein L Load (Protein A flow through); 4D: Protein L Eluate; 4E: Protein L flow through); Lanes 6A-6E: TrYbe 06 (6A: Protein A Load (Supernatant); 6B: Protein A Eluate; 6C: Protein L Load (Protein A flow through); 6D: Protein L Eluate; 6E: Protein L flow through). FIG. 7C: Densitometrical analysis of reducing SDS-PAGE. Samples include Protein A Eluates of TrYbe 04 and TrYbe 06 (horizontal axis). Analysis is displayed as a percentage relative to the density of the heavy chain band in the vertical axis.

FIG. 8: binding response (in RU for Response Units or Resonance Units; vertical axis) for each concentration (horizontal axis) of the test molecules and control over the commercial purified Protein A (FIG. 8A) and purified recombinant protein A (FIG. 8B).

### EXAMPLES

### Example 1: Production of an Improved Multi-Specific Antibody Format of the Invention, Example of A Fab-2xdsscFv (TrYbe)

### Gene Design and Expression in CHO-S XE Cell Line

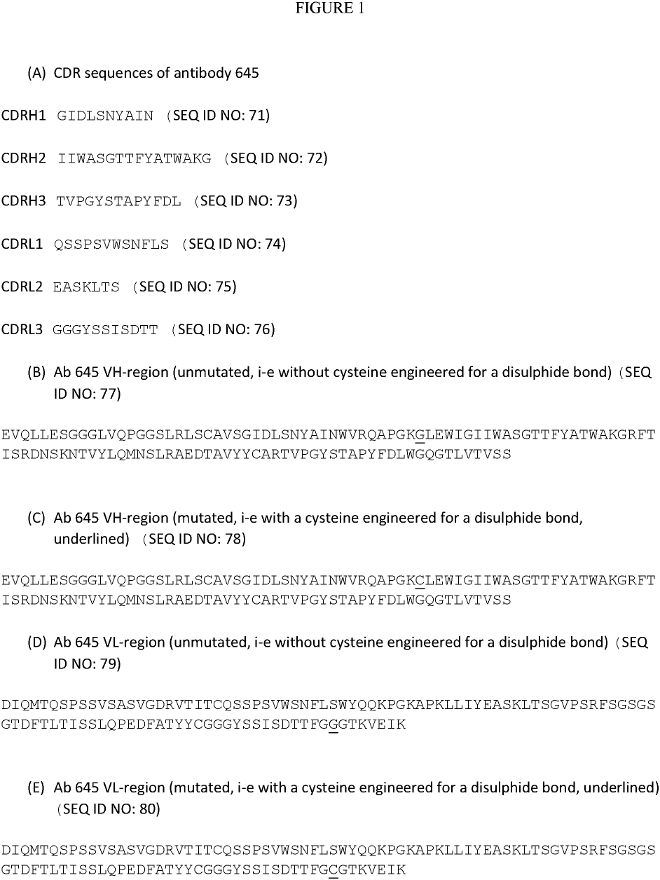

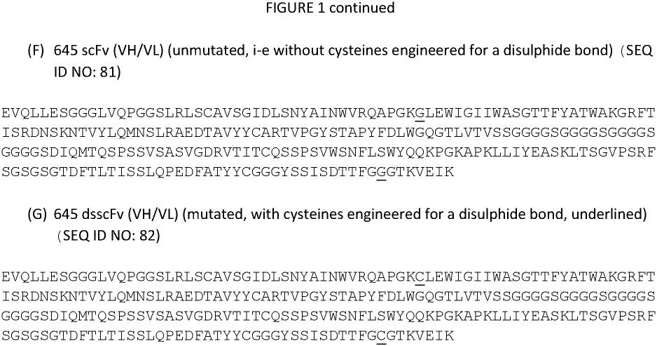

TrYbe antibody was designed with an anti-Antigen#1 (or “Ag#1”) V-region fixed in the Fab position; the anti-albumin(Antigen#2, or “Ag#2” in the following example) V-region (645gL4gH5) and Antigen#3 (or “Ag#3”) V-region (VH1) were reformatted into disulfide-stabilised scFv in the HL orientation (dsHL) and linked to the C-termini of the respective heavy and light chain constant regions via a 11 -amino acid glycine-serine rich linkers. The resulting antibody is referred to as Trybe 03. The sequences of anti-albumin 645 antibody are shown in FIG. 1.

The light chain and heavy chain genes were independently cloned into proprietary mammalian expression vectors for transient expression under the control of a hCMV promoter. Equal ratios of both plasmids were transfected into the CHO-S XE cell line (UCB) using the commercial ExpiCHO expifectamine transient expression kit (Thermo Scientific). The cultures were incubated in Corning roller bottles with vented caps at 37° C., 8.0% CO₂, 190 rpm. After 18-22 h, the cultures were fed with the appropriate volumes of CHO enhancer and feeds for the HiTiter method as provided by the manufacturer. Cultures were reincubated at 32° C., 8.0% CO₂, 190 rpm for an additional 10 to 12 days. The supernatant was harvested by centrifugation at 4000 rpm for 1 h at 4° C. prior to filter-sterilization through a 0.45 µm followed by a 0.2 µm filter. Expression titres were quantified by Protein G HPLC using a 1 ml GE HiTrap Protein G column (GE Healthcare) and Fab standards produced in-house. The expression titre was 160 mg/L.

### Purification of TrYbe 03 Using A Protein A Affinity Chromatography

The TrYbe 03 was purified by native protein A capture step followed by a preparative size exclusion polishing step. Clarified supernatants from standard transient CHO expression were loaded onto a MabSelect (GE Healthcare) column giving a 5 min contact time and washed with binding buffer (20 mM Hepes pH7.4 + 150 mM NaCl). Bound material was eluted with a 0.1 M sodium citrate pH3.1 step elution and neutralised with 2 M Tris/HCl pH8.5 and quantified by absorbance at 280 nm.

Size exclusion chromatography (SE-UPLC) was used to determine the purity status of the eluted product. The antibody (~2 µg) was loaded on to a BEH200, 200 Å, 1.7 µm, 4.6 mm ID x 300 mm column (Waters ACQUITY) and developed with an isocratic gradient of 0.2 M phosphate pH 7 at 0.35 mL/min. Continuous detection was by absorbance at 280 nm and multi-channel fluorescence (FLR) detector (Waters). The eluted TrYbe 03 antibody was found to be 72 % monomer.

The neutralised samples were concentrated using Amicon Ultra-15 concentrator (10 kDa molecular weight cut off membrane) and centrifugation at 4000xg in a swing out rotor. Concentrated samples were applied to a XK16/60 Superdex200 column (GE Healthcare) equilibrated in PBS, pH7.4 and developed with an isocratic gradient of PBS, pH7.4 at 1 ml/min. Fractions were collected and analysed by size exclusion chromatography on a BEH200, 200 Å, 1.7 µm, 4.6 mm ID x 300 mm column (Aquity) and developed with an isocratic gradient of 0.2 M phosphate pH 7 at 0.35 mL/min, with detection by absorbance at 280 nm and multi-channel fluorescence (FLR) detector (Waters). Selected monomer fractions were pooled, 0.22 µm sterile filtered and final samples were assayed for concentration by A280 Scanning on DropSense96 (Trinean). Endotoxin level was less than 1.0EU/mg as assessed by Charles River’s EndoSafe® Portable Test System with Limulus Amebocyte Lysate (LAL) test cartridges.

### Analysis by Size Exclusion Chromatography

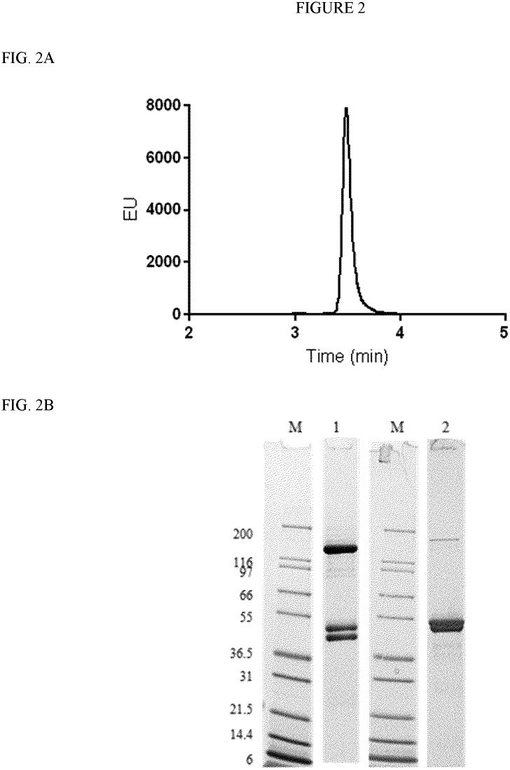

Monomer status of the final TrYbe 03 was determined by size exclusion chromatography on a BEH200, 200 Å, 1.7 µm, 4.6 mm ID x 300 mm column (Aquity) and developed with an isocratic gradient of 0.2 M phosphate pH 7 at 0.35 mL/min, with detection by absorbance at 280 nm and multi-channel fluorescence (FLR) detector (Waters). The final TrYbe 03 antibody was found to be >99 % monomeric. (FIG. 2A)

### SDS-PAGE Analysis

For analysis by sodium dodecyl sulfate-polyacrylamide gel electrophoresis (SDS-PAGE) samples were prepared by adding 4 x Novex NuPAGE LDS sample buffer (Life Technologies) and either 10X NuPAGE sample reducing agent (Life Technologies) or 100 mM N-ethylmaleimide (Sigma-Aldrich) to ~ 5 µg purified protein, and were heated to 100° C. for 3 min. The samples were loaded onto a 10 well Novex 4-20% Tris-glycine 1.0 mm SDS-polyacrylamide gel (Life Technologies) and separated at a constant voltage of 225 V for 40 min in Tris-glycine SDS running buffer (Life Technologies). Novex Mark12 wide-range protein standards (Life Technologies) were used as standards. The gel was stained with Coomassie Quick Stain (Generon) and destained in distilled water.

On non-reducing SDS-PAGE the TrYbe (lane 1), theoretical molecular weight (MW) of ~100 kDa, migrated to ~ 120 kDa (FIG. 2B). When the TrYbe protein was reduced (lane 2), both chains migrated at a mobility rate approaching their respective theoretical MWs, heavy chain (HC) ~52 kDa and light chain (LC) ~51 kDa. Additional bands on the non-reduced gel (lane 1) at ~45 - 50 kDa are ‘free’ LC and HC missing the disulphide bond in the Fab portion of the molecule, they do not migrate to the same position as the LC and HC in lane 2 as they are not fully reduced.

The present inventors have observed that Trybe 03 had improved properties over the multi-specific antibodies of the prior art, in particular in that it maximised the amount of proteins of interest (i-e the correct multi-specific antibody) obtained after a one-step purification on a protein A chromatography column. Indeed, previously, the inventors detected appended light chains unpaired with their corresponding heavy chains, co-purified with the multi-specific antibody of interest and which had a propensity to form dimers of appended light chains (appended LC dimers), which needed to be purified away by an additional capture step. Unexpectedly, after the protein A purification step, no light chain or LC dimer was detected as a by-product of the production process of TrYbe 03 and only the desired multi-specific antibody was eluted from the protein A column. In addition, the multi-specific antibody was highly monomeric.

The inventors made the hypothesis that the isolation and removal of the appended LC dimers occurred concurrently with the purification of Trybe 03.

To confirm this hypothesis, additional experiments, with alternative multi-specific antibody formats, were performed and are described in the following examples.

### Example 2: Production of Alternative Antibody Formats for Further Analysis in Examples 3 to 6

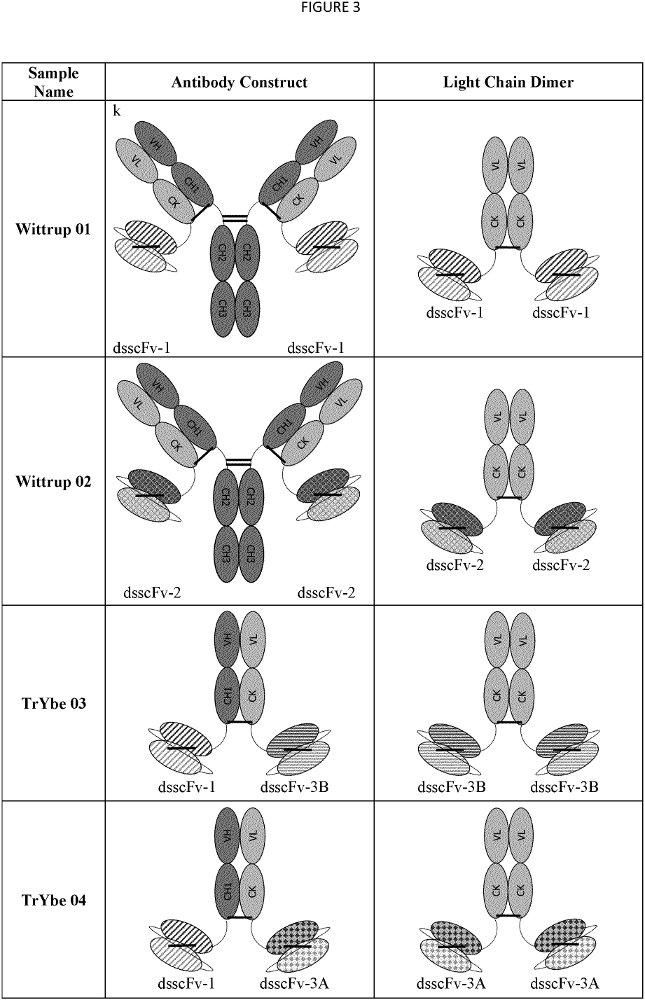

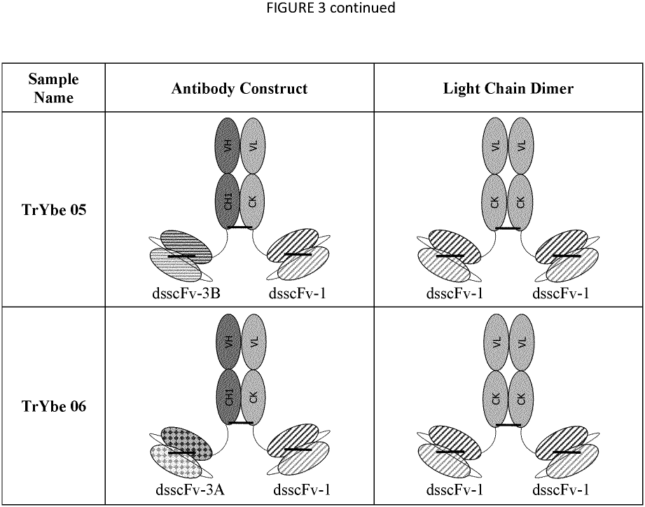

The constructs as illustrated in FIG. 3 were produced as described in Table 1 and below. All Wittrup molecules have a common heavy chain (hg1FL) and Fab region. All TrYbe molecules have a common Fab region.

TABLE 1

| | |
|---|---|
| Antibody construct | Description |
| WITTRUP 01 | Ag#1 hg1FL, |
| | Ag#1 Fab LC- Ag#2 dsscFv HL |
| WITTRUP 02 | Ag#1 hg1FL, |
| | Ag#1 Fab LC-Ag#4 dsscFv HL |
| TRYBE 03 | Ag#1 Fab, |
| | Ag#2 dsscFv HL (HC), |
| | Ag#3 dsscFv HL (LC) (VH1) |
| TRYBE 04 | Ag#1 Fab, |
| | Ag#2 dsscFv HL (HC), |
| | Ag#3 dsscFv HL (LC) (VH3) |
| TRYBE 05 | Ag#1 Fab, |
| | Ag#3 dsscFv HL (HC) (VH1), |
| | Ag#2 dsscFv HL (LC) |
| TRYBE 06 | Ag#1 Fab, |
| | Ag#3 dsscFv HL (HC) (VH3), |
| | Ag#2 dsscFv HL (LC) |

In the following examples, 645 gH5gL4 dsscFv(HL), i-e Ag#2 dsscFv HL, is termed dsscFv 1.

Ag#3 dsscFv HL (VH1), comprising a VH1 domain, is termed dsscFv 3B,

Ag#3 dsscFv HL (VH3), comprising a VH3 domain, is termed dsscFv 3A.

Ag#4 dsscFv HL is termed dsscFv 2.

### Transient Expression

Heavy and light chain antibody genes were independently cloned into proprietary mammalian expression vectors for transient expression under the control of a hCMV-mie promoter. Plasmids were transfected into a proprietary CHO-SXE cell line using the commercial ExpiCHO expifectamine transient expression kit (Thermo Scientific). The cultures were incubated in Corning roller bottles with vented caps at 37° C., 8.0% CO₂, 190 rpm. After 18-22 h, the cultures were fed with the appropriate volumes of CHO enhancer and feeds for the HiTiter method as provided by the manufacturer. Cultures were then incubated at 32° C., 8.0% CO₂, 190 rpm for an additional 10 to 12 days. The supernatant was harvested by centrifugation at 4000 rpm for 1 h at 4° C. prior to filter-sterilization through a 0.45 µm followed by a 0.2 µm filter.

Expression titres were quantified by Protein A HPLC and Protein L HPLC using either a 1 ml HiTrap Protein A column or a 1 ml HiTrap Protein L column (GE Healthcare). Columns were equilibrated in a phosphate buffer, 10 µl of sample was injected, column was washed, and an acidic step elution was used to elute the antibody. Concentrations were calculated using the elution peak area for each sample compared to a standard curve generated using in-house purified Fab standards with appropriate molar extinction co-efficient correction.

Protein L ligand binds via the VL domain, i-e the light chain of antibodies. Protein A binds the CH2/CH3 interface of the Fc and a selection of human VH domains comprising a protein A binding domain.

### Expression of Light Chain Plasmids Only

For expression of the light chains appended with a disulphide stabilised single chain Fv (LC-dsscFv), only the light chain plasmids were transfected, expressed and quantified by the above method. Table 1a lists the titres for these expressed light chain dimers as quantified by both Protein A and Protein L HPLC assays.

The quantification of LC-dsscFv-1 supernatant gave equivalent results in the Protein L and Protein A assays. In contrast, the LC-dsscFv-2 and LC-dsscFv-3B supernatants were quantifiable by Protein L but the Protein A assay was below the level of quantification. The quantification of the LC-dsscFv-3A expression gave a value of the Protein-A assay of about a third of the Protein L assay.

TABLE 1a

Quantification of expressed Light Chain Dimer by Protein A and Protein L HPLC assay. LOQ = Limit of quantification.

| Description of Light Chain dsscFv (Light Chain Dimer) | Protein A mg/L | Protein L mg/L |
|---|---|---|
| dsscFv-1 | 221.2 | 220.2 |
| dsscFv-2 | <LOQ | 250.5 |
| dsscFv-3B | <LOQ | 155.3 |
| dsscFv-3A | 43.9 | 120.5 |

TABLE 1b

Strength of Light Chain binding to Protein A. Binding strengths have been categorised as strong (++), weak (+), none (-).

| | | |
|---|---|---|
| Antibody Name | Description of Light Chain dsscFv | Protein A Binding |
| Wittrup 01 | dsscFv-1 | ++ |
| TrYbe 05 | | |
| TrYbe 06 | | |
| Wittrup 02 | dsscFv-2 | - |
| TrYbe 03 | dsscFv-3B | - |
| TrYbe 04 | dsscFv-3A | + |

As shown in Table 1b, LC-dsscFv-1 contains a dsscFv which binds Protein A, explaining why the calculated Protein L and Protein A titres were equivalent (Table 2a). At the contrary, LC-dsscFv-2 and LC-dsscFv-3B were only quantifiable by the Protein L assay and not the Protein A assay and it was confirmed that they do not comprise a protein A binding domain. It was observed that LC-dsscFv-3A contained a dsscFv that binds Protein A weakly, therefore the concentration calculated was only a third of the concentration from the Protein L assay.

Therefore, the results show that dsscFv-1 and dsscFv-3A comprise a protein A binding domain. In particular, dsscFv-3A comprises a VH3 domain which is able to bind protein A.

At the contrary, dsscFv-2 and dsscFv-3B do not bind protein A. In particular, dsscFv-3B comprises a VH1 domain which is unable to bind protein A.

### Co-Expression of Heavy Chain and Light Chain Plasmids

For the expression of antibody constructs, equal ratios of heavy and light chain plasmids were co-transfected and expressed by the above method. These antibodies share the same Fab region and isotype.

To ensure that the test supernatants studied in the following Examples (3, 4, 5 and 6) contained excess light chain, the corresponding light chain only supernatant was added to the antibody supernatant. The resulting test supernatants were quantified by Protein A and Protein L HPLC assays (Table 2a).

The quantification of Wittrup 01, TrYbe 05 and TrYbe 06 test supernatants gave equivalent results in both Protein A and Protein L assays. For Wittrup 02, TrYbe 03 and TrYbe 04 the concentration determined by Protein A assay was approximately half of that determined by the Protein L assay.

Wittrup 01, TrYbe 05 and TrYbe 06 share the same light chain, as described in Table 1b and Table 2b, this light chain has a Protein A binding dsscFv, so the calculated Protein L and Protein A titres were equivalent as both the antibody and light chain dimer can bind in both assays. The Protein A assay can be used to determine the concentration of Wittrup 02 and TrYbe 03 as the antibody can bind Protein A, however both have a non-protein A binding dsscFv on the light chain meaning that respective light chain dimers can only be quantified by the Protein L assay, thus accounting for the 2-fold difference between the two assays. TrYbe 04 has a weak Protein A binding dsscFv on the light chain, therefore only some of the light chain dimer binds and the concentration calculated was only half of the concentration from the Protein L assay.

TABLE 2a

Quantification of test material by Protein A and Protein L HPLC assay. Samples prepared by spiking light chain only supernatant into the respective antibody supernatants.

| | | | | |
|---|---|---|---|---|
| Sample Name | Chain | Description of appended dsscFv | Protein A mg/L | Protein L mg/L |
| Wittrup 01 | Heavy | - | 47.0 | 66.9 |
| | Light | dsscFv-1 | | |
| Wittrup 02 | Heavy | - | 97.2 | 180.2 |
| | Light | dsscFv-2 | | |
| TrYbe 03 | Heavy | dsscFv-1 | 95.8 | 153.4 |
| | Light | dsscFv-3B | | |
| TrYbe 04 | Heavy | dsscFv-1 | 129.6 | 219.1 |
| | Light | dsscFv-3A | | |
| TrYbe 05 | Heavy | dsscFv-3B | 137.6 | 143.0 |
| | Light | dsscFv-1 | | |
| TrYbe 06 | Heavy | dsscFv-3A | 280.9 | 296.2 |
| | Light | dsscFv-1 | | |

TABLE 2b

Strength of Light Chain binding to Protein A. Binding strengths have been categorised as strong (++), weak (+), none (-). All heavy chains are described as strong binders as they bind through the common Fab (Wittrup & TrYbe) or through the Fc (Wittrup only).

| | | | |
|---|---|---|---|
| Sample Name | Chain | Description of appended scFv | Protein A Binding |
| Wittrup 01 | Heavy | - | ++ |
| | Light | dsscFv-1 | ++ |
| Wittrup 02 | Heavy | - | ++ |
| | Light | dsscFv-2 | - |
| TrYbe 03 | Heavy | dsscFv-1 | ++ |
| | Light | dsscFv-3B | - |
| TrYbe 04 | Heavy | dsscFv-1 | ++ |
| | Light | dsscFv-3A | + |
| TrYbe 05 | Heavy | dsscFv-3B | - |
| | Light | dsscFv-1 | ++ |
| TrYbe 06 | Heavy | dsscFv-3A | + |
| | Light | dsscFv-1 | ++ |

### Example 3: Protein A Purification of Wittrup Antibody Formats; Selecting the dsscFv Variable Region With Appropriate Protein A Binding Properties

The test supernatants for both Wittrup molecules were prepared as described in Example 2, and contain both antibody and light chain dimer. These Wittrup antibodies share the same IgG component (Fc and Fab) but each has a different dsscFv appended to the light chain. Wittrup 01 has a Protein A binding dsscFv appended to the light chain whereas Wittrup 02 has a non-Protein A binding dsscFv appended to the light chain.

As shown in Example 2, the Wittrup 01 and Wittrup 02 test supernatants were quantified by Protein A and Protein L HPLC assays (Table 3a). Wittrup 01 gave approximately equivalent results in both assays, whereas for Wittrup 02 the Protein A assay was only half of the Protein L assay. Wittrup 01 has a Protein A binding dsscFv appended to the light chain (Table 3b), so the titres calculated by Protein L and Protein A are equivalent as both ligands can detect light chain dimers. The Protein A assay result for Wittrup 02, which has a non-protein A binding dsscFv appended to the light chain (Table 3b), is significantly lower than the Protein L assay as only the antibody can bind Protein A whereas both Wittrup antibody and light chain dimer can bind Protein L.

TABLE 3a

Quantification of test material by Protein A and Protein L HPLC assay. Samples prepared by spiking light chain only supernatant into the respective antibody supernatants.

| | | | | |
|---|---|---|---|---|
| Sample Name | Chain | Description of appended scFv | Protein A mg/L | Protein L mg/L |
| Wittrup 01 | Heavy | - | 47.0 | 66.9 |
| | Light | dsscFv-1 | | |
| Wittrup 02 | Heavy | - | 97.2 | 180.2 |
| | Light | dsscFv-2 | | |

TABLE 3b

Strength of Light Chain binding to Protein A. Binding strengths have been categorised as strong (++), weak (+), none (-).

| | | | |
|---|---|---|---|
| Sample Name | Chain | Description of appended scFv | Protein A Binding |
| Wittrup 01 | Heavy | - | ++ |
| | Light | dsscFv-1 | ++ |
| Wittrup 02 | Heavy | - | ++ |
| | Light | dsscFv-2 | - |

### Protein A Purification

The test supernatants were loaded onto a MabSelect (GE Healthcare) column with a 15 min contact time and washed with binding buffer (200 mM glycine, pH7.5). The flow through was collected and 0.22 µm sterile filtered. Bound material was eluted with a 0.1 M sodium citrate pH3.2 step elution, the elution peak was collected, neutralised with 2 M Tris-HCl pH8.5 and the purified protein was quantified by absorbance at 280 nm. To confirm that the protein was completely eluted from the column a second elution with 0.1 M Citrate pH2.1 was performed.

### Protein L Purification

The flow throughs from the Protein A purifications were loaded onto a Protein L (GE Healthcare) column with a 10 min contact time and washed with binding buffer (200 mM glycine, pH7.5). The flow through was collected and 0.22 µm sterile filtered. Bound material was eluted with a 0.1 M Glycine/HCl pHy step elution, the elution peak was collected, neutralised with 2 M Tris-HCl pH8.5 and the purified protein was quantified by absorbance at 280 nm. To confirm that the protein was completely eluted from the column a second elution with 0.1 M Citrate pH2.1 was performed.

### SDS-PAGE

For analysis by sodium dodecyl sulfate-polyacrylamide gel electrophoresis (SDS-PAGE) samples were prepared by adding 4 x Novex NuPAGE LDS sample buffer (Life Technologies) and either 10X NuPAGE sample reducing agent (Life Technologies) or 100 mM N-ethylmaleimide (Sigma-Aldrich), and were heated to 100° C. for 3 min. The samples were loaded onto a 15 well Novex 4-20% Tris-glycine 1.0 mm SDS-polyacrylamide gel (Life Technologies) and separated at a constant voltage of 225 V for 40 min in Tris-glycine SDS running buffer (made in-house). Novex Mark12 wide-range protein standards (Life Technologies) were used as molecular weight markers. The gel was stained with Coomassie Quick Stain (Generon) and destained in distilled water.

### Results

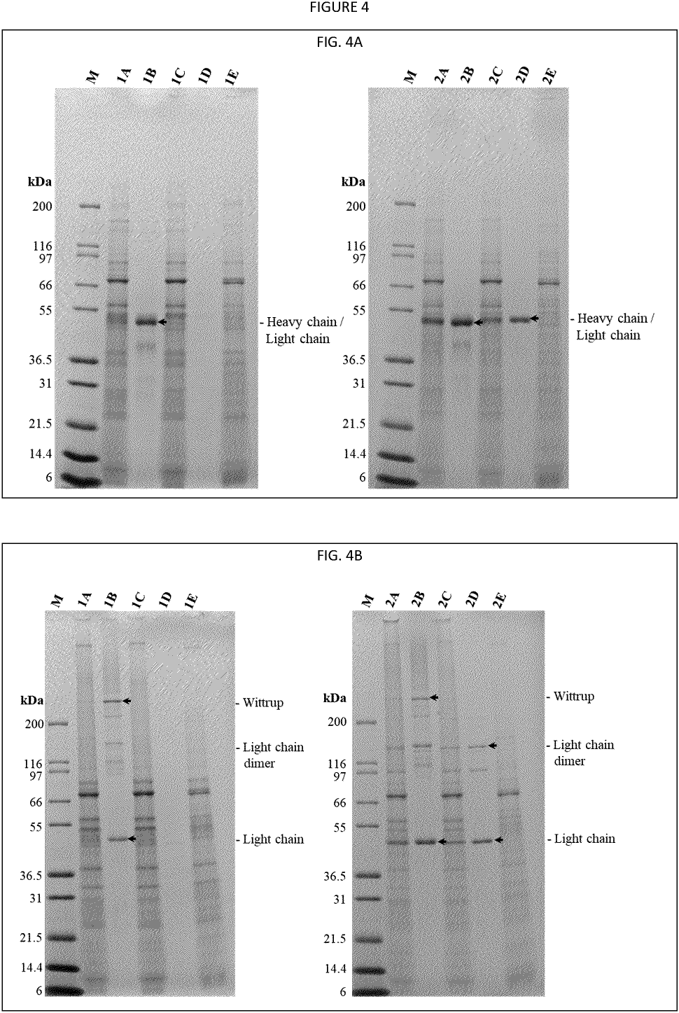

To evaluate sequential Protein A and Protein L purifications, reduced (FIG. 4A) and non-reduced (FIG. 4B) samples were prepared for SDS-PAGE analysis. These samples included Protein A load material, Protein A eluate, Protein L load material (Protein A flow through), Protein L eluate and Protein L flow through.

Wittrup 01 has a Protein A binding dsscFv appended to the light chain. In the reduced Protein A eluate (lane 1B) there is one band as the heavy and light chains are similar in size and therefore co-migrate to the same position. In the Protein L eluate (lane 1D) there are no detectable bands. This indicates that the light chain dimer was co-purified with the Wittrup 01 antibody during the Protein A purification. In contrast, Wittrup 02 has a non-Protein A binding dsscFv appended to the light chain. The Protein A eluate (lane 2B) looks comparable to the Wittrup 01 Protein A eluate but in the Protein L eluate there is a light chain band present indicating that the light chain dimer was not captured in the Protein A purification but flowed through the column and was subsequently captured in the Protein L purification.

On the non-reduced gel for Wittrup 01, there are bands for the Wittrup antibody and the light chain dimer in the Protein A eluate (lane 1B). There are also additional bands present in this lane due to incomplete formation of the natural interchain disulphide (ds) bond between the CH1 and C_{K} in a portion of the molecules. The Protein L eluate (lane 1D) has no detectable bands again showing that the light chain dimer co-purified with the Wittrup 01 in the Protein A purification. For Wittrup 02, there is a Wittrup band in the Protein A eluate (lane 2B) as well as the additional bands due to incomplete disulphide formation. The light chain dimer band can be seen in both the Protein L load and the Protein L Eluate (lane 2C, lane 2D) but not in the Protein A eluate. This further indicates that only the Wittrup 02 antibody was captured in the Protein A purification and that the light chain dimer flowed through the column and was subsequently captured in the Protein L purification.

In summary, the presence of a dsscFv able to bind Protein A appended to the light chain in the Wittrup antibody resulted in the co-purification of the light chain dimers, which could be avoided by selecting a dsscFv unable to bind protein A appended to the light chain of the Wittrup format. Therefore, the inventors provided an improved multi-specific antibody wherein the light chain may be selected or engineered to be a non-Protein A binder.

### Example 4: Protein A Purification of TrYbe Antibody Formats, With Different Variable Region Grafting; Framework Selection for Appropriate Protein A Binding Properties of the Light Chain Appended DsscFv

The test supernatants for both TrYbe 03 and 04 molecules were prepared as described in Example 2 and contain both antibody and light chain dimer. These TrYbes share the same Fab and the same Protein A binding dsscFv appended to the heavy chain. The light chain appended dsscFvs are derived from the same parent variable region but in TrYbe 03 the CDRs were grafted onto a non-Protein A binding framework (VH1 domain) whereas in TrYbe 04 the CDRs were grafted onto a Protein A binding framework (VH3 domain).

The TrYbe 03 and TrYbe 04 test supernatants were quantified by Protein A and Protein L HPLC assays (Table 4a) and in both cases the Protein A assay is lower than the Protein L assay.

The concentration of TrYbe 03 as determined by Protein A assay is about half of the Protein L assay, as this TrYbe has a non-Protein A binding dsscFv on the light chain (Table 4b) only the TrYbe antibody can bind Protein A whereas both the TrYbe 03 and light chain dimer can be quantified by the Protein L assay. TrYbe 04 has a weak Protein A binding dsscFv on the light chain (Table 4b), all the TrYbe and light chain dimer can bind to the Protein L assay, but the Protein A assay binds all the TrYbe and only a proportion of the light chain dimer. Therefore, it is not possible to accurately quantify the total light chain dimer and TrYbe by Protein A in this situation.

TABLE 4a

Quantification of test supernatants by Protein A and Protein L HPLC assay. Samples prepared by spiking light chain only supernatant into the respective antibody supernatants.

| | | | | |
|---|---|---|---|---|
| Sample Name | Chain | Description of appended scFv | Protein A mg/L | Protein L mg/L |
| TrYbe 03 | Heavy | dsscFv-1 | 95.8 | 153.4 |
| | Light | dsscFv-3B | | |
| TrYbe 04 | Heavy | dsscFv-1 | 129.6 | 219.1 |
| | Light | dsscFv-3A | | |

TABLE 4b

Strength of Light Chain binding to Protein A. Binding strengths have been categorised as strong (++), weak (+), none (-).

| | | | |
|---|---|---|---|
| Sample Name | Chain | Description of appended scFv | Protein A Binding |
| TrYbe 03 | Heavy | dsscFv-1 | ++ |
| | Light | dsscFv-3B | - |
| TrYbe 04 | Heavy | dsscFv-1 | ++ |
| | Light | dsscFv-3A | + |

### Protein A and Protein L Purification Steps, and SDS PAGE Analysis Were Done As Described Above in Example 3

### Densitometry

A Densitometrical analysis was performed on the reduced SDS-PAGE using ImageQuant image analysis software (GE Healthcare). Analysis is displayed as a percentage relative to the density of the heavy chain band.

### Results

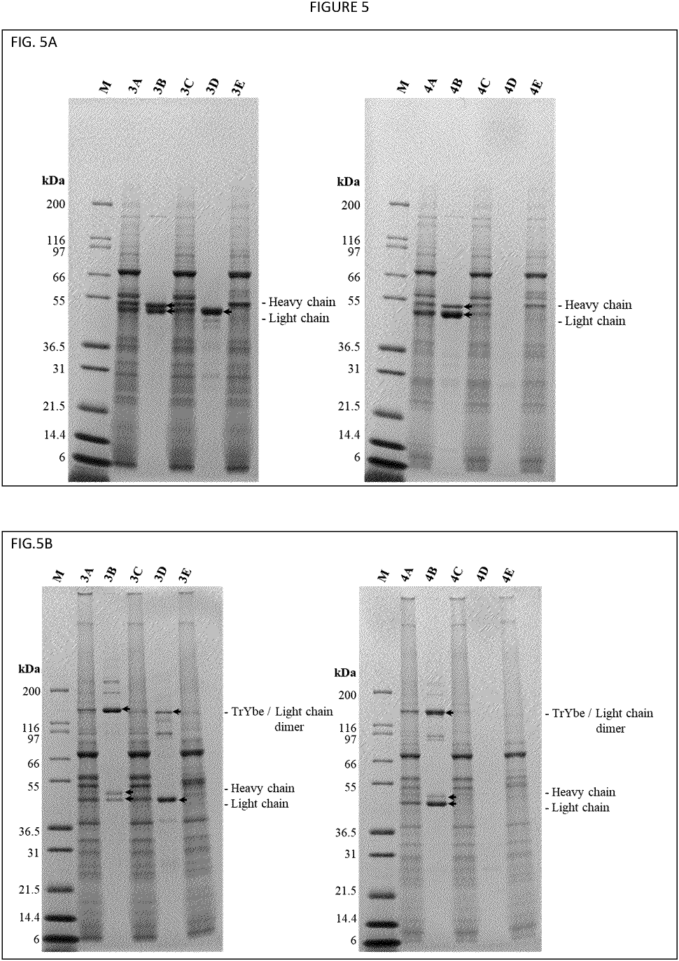

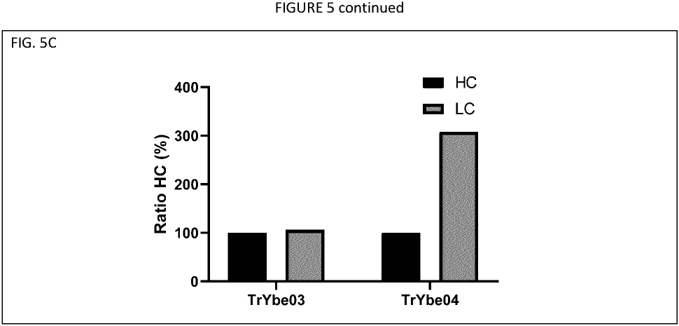

To evaluate the sequential Protein A and Protein L purifications, reduced (FIG. 5A) and non-reduced (FIG. 5B) samples were prepared for SDS-PAGE analysis. These samples included Protein A load material, Protein A eluate, Protein L load material (Protein A flow through), Protein L eluate and Protein L flow through. In addition, densitometrical analysis was performed on the reduced Protein A eluates to compare the proportions of heavy and light chains present (FIG. 5C).

TrYbe 03 has a non-Protein A binding dsscFv appended to the light chain. On the reduced gel in the Protein A eluate (lane 3B), there are two bands corresponding to the heavy and light chains; and in the Protein L eluate (lane 3D) only the light chain band is present. Densitometrical analysis showed the ratio of heavy and light chains present in the protein A eluate is equal. Therefore, only TrYbe 03 was captured by the Protein A purification and that the light chain dimer flowed through the column and was subsequently captured by the Protein L purification. In contrast, TrYbe 04 has a Protein A binding dsscFv appended to the light chain. On the reduced gel, in the Protein A eluate (lane 4B) there is a more intense light chain and less intense heavy chain. Densitometry (FIG. 5C) showed there to be three times more light chain than heavy chain present. In the Protein L eluate (lane 4D) there are no bands. This shows that the light chain dimer was co-purified with the TrYbe 04 during the Protein A purification. In Table 4b, TrYbe 04 is described as having a weak Protein A binding dsscFv appended to the light chain, this makes it hard to quantify by Protein A HPLC assay. However, under the conditions used for the preparative Protein A chromatography, the binding strength is sufficient and it is able to bind well to Protein A.

On the non-reduced gel, for TrYbe 03 there is a TrYbe band in the Protein A eluate (lane 3B) and a light chain dimer band in the Protein L eluate (lane 3D), they are similar in size, so the bands migrate to the same position. There are also heavy and light chain bands in the Protein A eluate and a light chain band in the Protein L eluate, this is due to the incomplete formation of the natural interchain disulphide (ds) bond between the CH1 and C_{K} in a small proportion of the molecules. This is also evident in the Protein L eluate (lane 3E) as there is non-ds bonded light chain present. Again, these observations indicate that only the TrYbe 03 antibody was captured by the Protein A purification and that the light chain dimer flowed through the column and was subsequently captured by the Protein L purification. For TrYbe 04, in the Protein A eluate (lane 4B) the TrYbe and light chain dimer bands co-migrate to the same position as they are similar in size. There are also heavy and light chain bands present due to incomplete interchain ds bond formation and there is more non-ds bonded light chain as the ds bond formation between two CK is less efficient than for the CH1/C_{K} pairing. Again, there are no bands in the Protein L eluate (lane 4D) indicating the light chain dimer was co-purified with the TrYbe 04 during the Protein A purification.

In summary, the presence of a Protein A binding graft of this dsscFv on the light chain resulted in the co-purification of light chain dimer with TrYbe. The same dsscFv was grafted onto a non-Protein A binding framework, then light chain dimer was not captured and only the TrYbe was purified by the Protein A chromatography.

Therefore, the inventors provided an improved multi-specific antibody wherein the VH framework of the dsscFv appended to the light chain was selected to be a non-Protein A binder. In the present case, a VH1 was selected for its inability to bind protein A. It will be understood by the skilled person that the same results can be obtained by selecting frameworks that do not bind protein A, for example a VH1, a VH2, a VH4, a VH5, a VH6, a naturally occurring VH3 unable to bind protein A, or a variant of a naturally occurring VH3 able to bind protein A, comprising at least one mutation abolishing its ability to bind protein A.

### Example 5: Protein A Purification of TrYbe Antibody Formats, With Alternate DsscFv Positioning for Appropriate Protein A Binding Properties of the Light Chain Appended DsscFv

The test supernatants for both TrYbe 03 and TrYbe 05 molecules were prepared as described in Example 2 and contain both antibody and light chain dimer. These TrYbe share the same Fab and the same pair of dsscFvs but the dsscFvs were appended onto opposite Fab chains. In TrYbe 03 the Protein A binding dsscFv is appended to the heavy chain and the non-Protein A binding dsscFv is appended to the light chain. Alternatively, in TrYbe 05 the Protein A binding dsscFv is appended to the light chain and the non-Protein A binding dsscFv is appended to the heavy chain.

The TrYbe 03 and TrYbe 05 test supernatants were quantified by Protein A and Protein L HPLC (Table 5a). For TrYbe 03 the Protein A assay is significantly lower than the Protein L assay whereas TrYbe 05 gives equivalent results in both assays.

The Protein A assay result for TrYbe 03 is significantly lower than the Protein L assay as this TrYbe has a non-Protein A binding dsscFv on the light chain (Table 5b) meaning that only the TrYbe antibody can bind Protein A, whereas both the TrYbe and light chain dimer can bind the Protein L assay. TrYbe 05 has a Protein A binding dsscFv appended to the light chain (Table 5b), so the calculated Protein L and Protein A titres are equivalent as both assays can bind TrYbe and light chain dimers.

TABLE 5a

Quantification of test supernatants by Protein A and Protein L HPLC assay. Samples prepared by spiking light chain only supernatant into the respective antibody supernatants.

| | | | | |
|---|---|---|---|---|
| Sample Name | Chain | Description of appended scFv | Protein A mg/L | Protein L mg/L |
| TrYbe 03 | Heavy | dsscFv-1 | 95.8 | 153.4 |
| | Light | dsscFv-3B | | |
| TrYbe 05 | Heavy | dsscFv-3B | 137.6 | 143.0 |
| | Light | dsscFv-1 | | |

TABLE 5b

Strength of Light Chain binding to Protein A. Binding strengths have been categorised as strong (++), weak (+), none (-).

| | | | |
|---|---|---|---|
| Sample Name | Chain | Description of appended scFv | Protein A Binding |
| TrYbe 03 | Heavy | dsscFv-1 | ++ |
| | Light | dsscFv-3B | – |
| TrYbe 05 | Heavy | dsscFv-3B | – |
| | Light | dsscFv-1 | ++ |

### Protein A and Protein L Purification Steps Were Performed as Described Above. SDS PAGE and Densitometrical Analyses Were Also Performed As Described Above

### Results

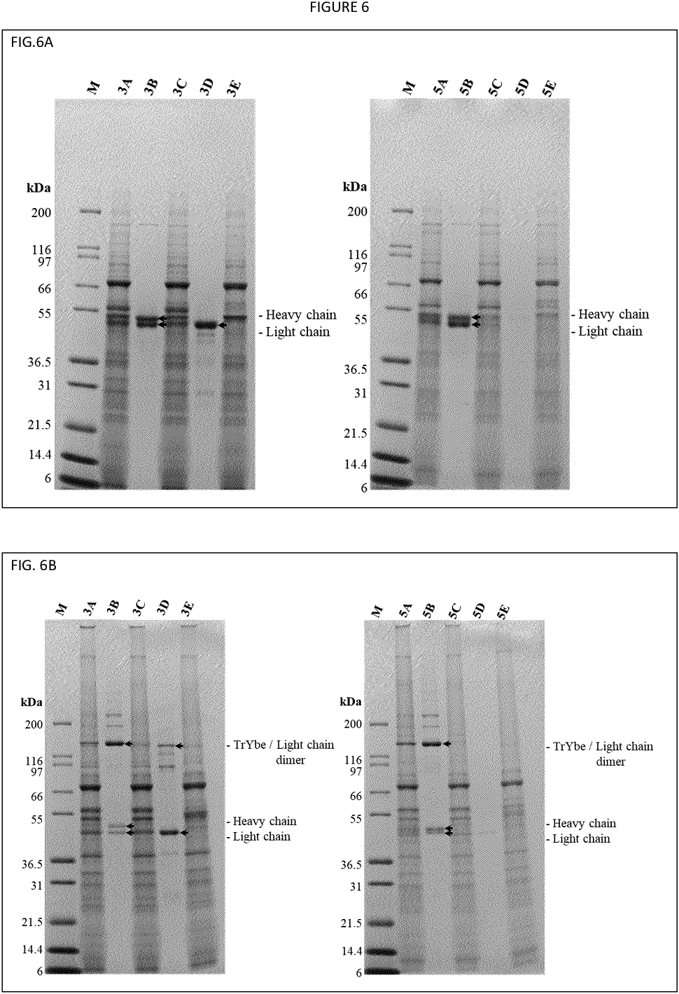

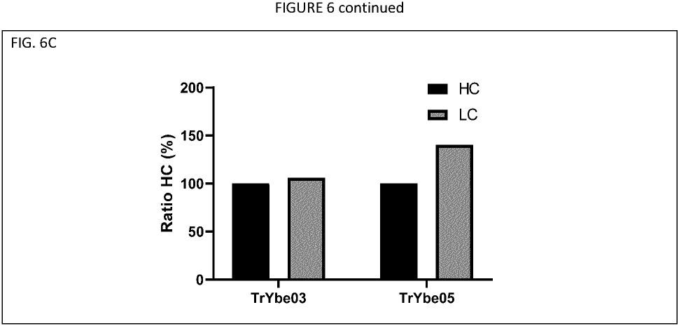

To evaluate the sequential Protein A and Protein L purifications, reduced (FIG. 6A) and non-reduced (FIG. 6B) samples were prepared for SDS-PAGE analysis. These samples included Protein A load material, Protein A eluate, Protein L load material (Protein A flow through), Protein L eluate and Protein L flow through. In addition, densitometrical analysis was performed on the reduced Protein A eluates to compare proportions of heavy and light chains present (FIG. 6C).

TrYbe 03 has the non-Protein A binding dsscFv appended to the light chain. On the reduced gel, in the Protein A eluate (lane 3B), there are two bands corresponding to the heavy and light chains, and in the Protein L eluate (lane 3D) only the light chain band is present. Densitometrical analysis shows the ratio of heavy and light chains present in the protein A eluate is equal. Therefore, only the TrYbe 03 was captured in the Protein A purification and the light chain dimer flowed through the column and was subsequently captured in the Protein L purification. In contrast, TrYbe 05 has the Protein A binding dsscFv appended to the light chain. In the reduced Protein A eluate (lane 5B) there is 40% more light chain than heavy chain present, and in the Protein L eluate (lane 5D) there are no detectable bands. This indicates that the light chain dimer was co-purified with the TrYbe 05 during the Protein A purification.

On the non-reduced gel, for TrYbe 03 there is a TrYbe band in the Protein A eluate (lane 3B) and a light chain dimer band in the Protein L eluate (lane 3D), they are similar in size, so the bands migrate to the same position. There are also heavy and light chain bands in the Protein A eluate and a light chain band in the Protein L eluate. These are due to the incomplete formation of the natural interchain disulphide (ds) bond between the CH1 and C_{K} in a small proportion of the molecules, or the corresponding C_{K}/C_{K} interchain disulphide in the light chain dimer. Again, these results indicate that only TrYbe was captured in the Protein A purification and that the light chain dimer flowed through the column and was subsequently captured in the Protein L purification. In the TrYbe 05 Protein A eluate (lane 5B) the TrYbe and light chain dimer bands co-migrate to the same position as they are very similar in size. Again, heavy and light chains due to non-formation of interchain disulphide binds are present as in lane 3B. In addition, there are also no detectable bands in the Protein L eluate (lane 5D) further indication that the light chain dimer was co-purified with the TrYbe during the Protein A purification.

In summary, the arrangement of the TrYbe molecule such that a Protein A binding dsscFv was appended to the light chain and a non-Protein A binding dsscFv was appended to the heavy chain resulted in the co-purification of both light chain dimer and TrYbe. By reversing this design and swapping the two dsscFvs such that the Protein A binding dsscFv was on the heavy chain and the non-Protein A binding dsscFv was on the light chain, the inventors showed that it was possible to purify only the TrYbe by Protein A affinity chromatography with the light chain dimer flowing through the column.

### Example 6: Protein A Purification of TrYbe Antibody Formats, With Inappropriate ScFv Selection for Protein A Binding Properties of the Light Chain Appended ScFv

The test supernatants for both TrYbe molecules were prepared as described in Example 1 and contain both antibody and light chain dimer. These TrYbes share the same Fab and the same pair of dsscFvs but the dsscFvs were appended onto the opposite Fab chains. Both dsscFvs bind Protein A but with different strengths. In TrYbe 04, the weaker Protein A binding dsscFv is appended to light chain and the strong Protein A binding dsscFv is appended to the heavy chain. Alternatively, in TrYbe 06, the weaker Protein A binding dsscFv is appended to the heavy chain and the strong Protein A binding dsscFv is appended to the light chain.

The TrYbe 04 and TrYbe 06 test supernatants were quantified by Protein A and Protein L HPLC (Table 6a). For TrYbe 06, the Protein A and Protein L assays gives equivalent results, whereas for TrYbe 04 the Protein A assay is lower than the Protein L assay.

TrYbe 06 has a strong Protein A binding dsscFv appended to the light chain (Table 7b), so the concentrations calculated for both the Protein L and Protein A assays are equivalent as both TrYbe and the light chain dimer can bind to both assays. TrYbe 04 has a weak Protein A binding dsscFv on the light chain (Table 6b), therefore all the TrYbe and only a proportion of the light chain dimer will bind to the Protein A assay. In contrast both TrYbe and light chain dimer bind fully to the Protein L assay. It is therefore not possible to fully quantify all the light chain dimer present in this test supernatant using the Protein A assay.

TABLE 6a

Quantification of test supernatants by Protein A and Protein L HPLC assay. Samples prepared by spiking light chain only supernatant into the respective antibody supernatants.

| | | | | |
|---|---|---|---|---|
| Sample Name | Chain | Description of appended scFv | Protein A mg/L | Protein L mg/L |
| TrYbe 04 | Heavy | dsscFv-1 | 129.6 | 219.1 |
| | Light | dsscFv-3A | | |
| TrYbe 06 | Heavy | dsscFv-3A | 280.9 | 296.2 |
| | Light | dsscFv-1 | | |

TABLE 6b

Strength of Light Chain binding to Protein A. Binding strengths have been categorised as strong (++), weak (+), none (-).

| | | | |
|---|---|---|---|
| Sample Name | Chain | Description of appended scFv | Protein A Binding |
| TrYbe 04 | Heavy | dsscFv-1 | ++ |
| | Light | dsscFv-3A | + |
| TrYbe 06 | Heavy | dsscFv-3A | + |
| | Light | dsscFv-1 | ++ |

### Protein A and Protein L Purification Steps Were Performed as Described Above. SDS PAGE and Densitometrical Analyses Were Also Performed As Described Above

### Results

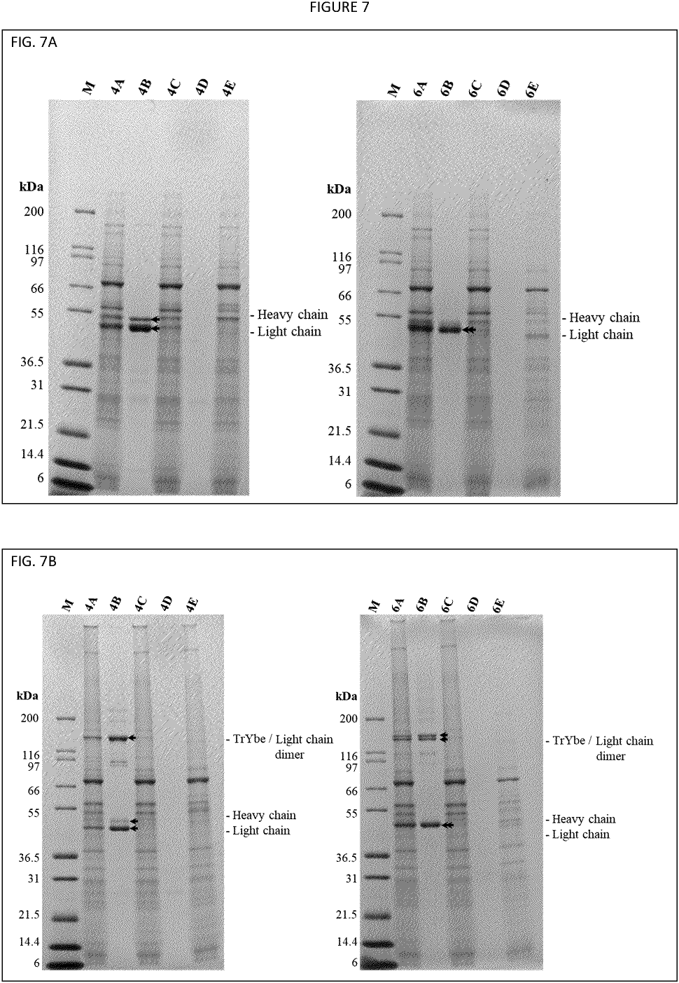

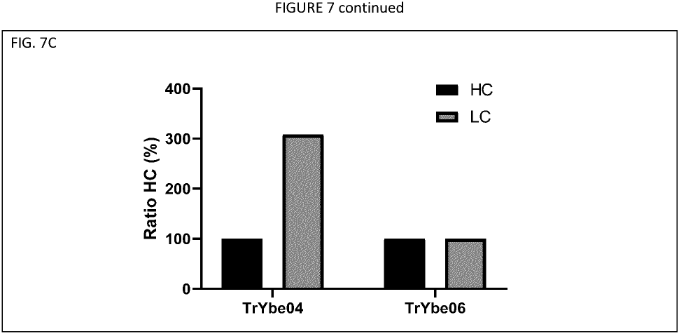

To evaluate the sequential Protein A and Protein L purifications, reduced (FIG. 7A) and non-reduced (FIG. 7B) samples were prepared for SDS-PAGE analysis. These samples included Protein A load material, Protein A eluate, Protein L load material (Protein A flow through), Protein L eluate and Protein L flow through. In addition, densitometrical analysis was performed on the reduced protein A eluates to compare proportions of heavy and light chains present (FIG. 7C).

TrYbe 04 has the weaker Protein A binding dsscFv appended to the light chain. On the reduced gel, in the Protein A eluate (lane 4B) there is a more intense light chain and less intense heavy chain. Densitometry showed there to be three times more light chain than heavy chain present. In the Protein L eluate (lane 4D) there are no bands. This indicates that the light chain dimer has co-purified with the TrYbe 04 during the Protein A purification. TrYbe 06 has a strong Protein A binding dsscFv appended to the light chain. In the reduced Protein A eluate (lane 6B) there is one band for both the heavy and light chain as in this example the bands co-migrate. There are no detectable bands in in the protein L eluate (lane 6D). As for TrYbe 06 this suggests that the light chain dimer has co-purified with the TrYbe during the Protein A purification.

For TrYbe 04, in the non-reduced Protein A eluate (lane 4B) the TrYbe and light chain dimer bands co-migrate to the same position as they are similar in size. There are also heavy and light chain bands due to incomplete interchain ds bond formation. There is more light chain due to the presence of light chain dimer and because the interchain disulphide bond formation between two C_{K} is less efficient than for the CH1/C_{K} pairing.

Like TrYbe 04, the Protein A eluate (lane 6B) for TrYbe 06, in the non-reduced gel, contains the TrYbe and light chain dimer however in this case there are two bands as they migrate slightly differently. There are also heavy and light chain bands but in contrast to the reduced gel they co-migrate so only one band is evident. As before, there are no bands in the Protein L eluate for either TrYbe 04 or TrYbe 06 (lane 4D, lane 6D) indicating the light chain dimer was co-purified with the TrYbe during the Protein A purifications.

In Summary, the presence of a Protein A binding dsscFv appended to the light chain resulted in co-purification of light chain dimer with TrYbe. This co-purification occured even when the light chain appended dsscFv was only a weak binder of Protein A. Therefore, the inventors showed the importance to completely abolish the ability of the antibody LC to bind protein A.

### Example 7: Protein-A Interaction Assay

A new method has been developed to qualitatively test antibody fragments for Protein-A binding through an interaction assay.

The assay consists of four key stages: load, wash, elution, re-equilibration. A 100 µl 2.1 x 30 mm POROS™ A 20 µm Column (Thermo Fisher Scientific, Waltham, MA) was equilibrated in running buffer (PBS pH 7.4). 50 µl of 1 mg/ml of test molecules or control molecules were loaded onto the column at 0.2 ml/min using an Agilent 1100 high-performance liquid chromatography (HPLC) system (Palo Alto, CA). Then, the column was washed slowly over 60 column volumes with a running buffer, such as PBS pH 7.4 for 30 minutes before applying an acidic step elution with 0.1 M Glycine-HCl pH 2.7 at 2.0 ml/min, for 2 minutes to remove any residual strong binders. Finally, the column was re-equilibrated in the running buffer (e.g. 50 CV PBS pH 7.4 at a flow-rate of 2.0 ml/min and a further 10 CV at 0.2 ml/min) in preparation for the next injection. Absorbance was read at 280 nm (A280).

### Test Molecules

Test molecules must be monovalent and monomeric, in this case purified BYbe (Fab-dsscFv) molecules with a murine Fab (which does not bind protein A) and dsscFv test V-regions appended to the heavy chain (HC) were used. dsscFv-1, dsscFv-2, dsscFv-3A, dsscFv-3B correspond to the dsscFv molecules used in the previous examples. In addition, dsscFv-4 was used, which comprises VH and VL regions corresponding to those of the hFab-4 binding fragment known to be a strong binder.

### Control Molecules

Control molecules have been used to ensure that the results were accurate. hFab-1 is a human Fab known to be a moderate binder. hFab-4 is a human Fab known to be a strong binder. mu Fab is a murine Fab and does not bind protein A. IgG bind Protein A strongly so an irrelevant IgG was used as control. Finally, human serum albumin (HSA) was used as a negative control.

### Results

The retention times are presented in Table 7. In this Protein A Interaction assay, Protein A non-binders can be defined where the main peak elutes in the Flow Through and therefore has a retention time which is inferior to 0.9 minutes. Peak retention times for weak to strong Protein A binders will range from 1-30 minutes respectively. It can also be expected that for stronger binders the peak shape will broaden as the molecule tumbles down the column. Strong binders may remain bound until the acidic elution step, where a peak at 31 minutes can be observed.

IgG’s bind Protein A strongly and so the IgG control was only eluted from the column during the acidic step of the assay and so the main peak retention time was 31 minutes. In contrast, the HSA negative control flew straight through the column and thus the main peak has a retention time of 0.7 minutes. The mu Fab used in the Fab-dsscFv test molecules has a main peak retention time <0.9 minutes, therefore we were confident that binding of the test molecules to Protein A occurred only through the dsscFv appended to the heavy chain of the Fab.

For all Protein A binding V-regions the retention time of the main peak was > 1 minute. The dsscFv-3A was previously described as a weak Protein A binder and has the shortest retention time at only 1.8 minutes.

Other Protein A binding V-regions (dsscFv-1, dsscFv-4) had later retention times indicating they are stronger binders than dsscFv-3A.

For Protein A non-binding V-regions (dsscFv-2, dsscFv-3B, dsscFv-mul) the Fab-dsscFv flew straight through the column and the retention time of the main peak is <0.9 minutes.

TABLE 7

Retention times obtained observed in the Protein A interaction assay

| Assay step: | FT | Wash | Elution |
|---|---|---|---|
| Binding Strength: | None | Weaker > > > > > > Stronger | Strong |
| Retention time: | 0-1 min | 1-30 min | 31 min |
| mu Fab, dsscFv-1 | | 3.584 | |
| mu Fab, dsscFv-2 | 0.797 | | |
| mu Fab, dsscFv-3A | | 1.818 | |
| mu Fab, dsscFv-3B | 0.781 | | |
| mu Fab, dsscFv-4 | | 30.073 | |
| mu Fab, ds scFv-mu1 (negative control) | 0.835 | | |
| hFab-1 (moderate binder control) | | 5.342 | |
| hFab-4 (strong binder control) | | 30.107 | |
| mu Fab (non-binder control) | 0.798 | | |
| hu IgG (positive control) | | | 31.275 |

### Example 8: Biacore Assay

In order to confirm the ability of an antibody construct comprising either natural or engineered Variable regions to bind protein A, the binding can be measured by Surface Plasmon resonance (SPR), in particular using Biacore.

SPR is a commonly used technology for detailed and quantitative studies of protein-protein interactions. It is often used to determine their equilibrium and kinetic parameters (Hashimoto, 2000). A Biacore method has been established to quantitatively assess the binding of antibody test molecules (such as BYbes) to Protein A. A BIAcore™ T200 instrument (GE Healthcare) was used to carry out the SPR experiments.

Binding to two forms of native Protein A was assessed: a commercially sourced Protein A purified from S.aureus (Sigma Aldrich), and a recombinant purified form (prepared in-house). Each were immobilised by standard amine coupling chemistry to a CM5 sensor chip surface (GE Healthcare) to a level of approximately 400RU. After which the binding of the test molecules was assessed by titrating each over the chip surface using a 60 s injection at 30 µl/min. HBS-EP+ (10 mM HEPES, 150 mM NaCl, 3 mM EDTA and 0.05 % Polysorbate 20) used as both sample dilute and running buffer, Between each injection, the surface was regenerated using a 60 s injection (at 10 µ1/min) injection of 10 mM glycine pH 1.7. Each sample was titrated over a 10-point concentration series in 3-fold dilutions from the highest concentration achievable dependent on the stock concentration (90, 30 or 10 µM) with a 0 nM blank injection was included for each sample to subtracted instrument noise and drift.

Mouse Fab samples fused to dsscFv sequences were selected as described in the previous example. In addition, the Mu Fab, dsscFv-mu1 was used as a negative control, comprising negative control mouse sequences with known absence of protein A binding.

### Results

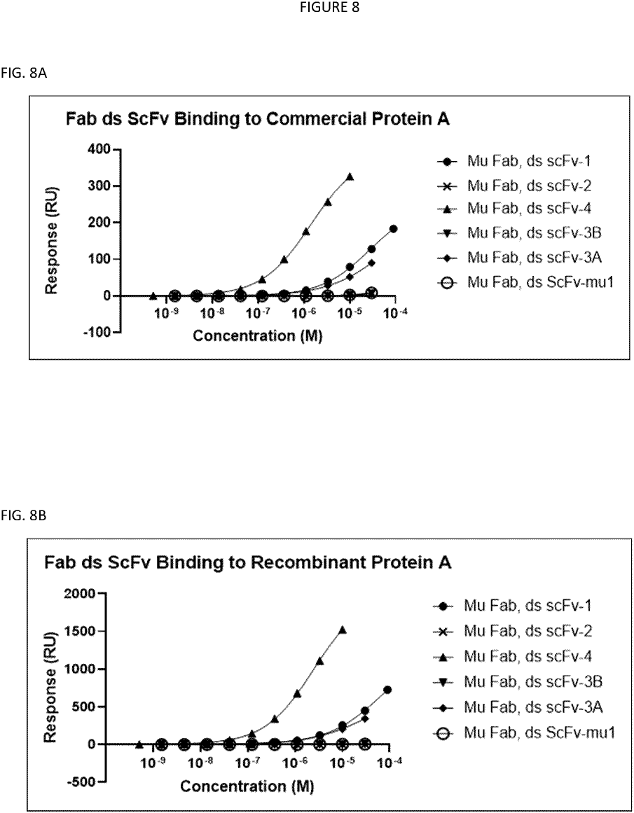

Tables 8a and 8b, and FIG. 8 represent the binding response at the end of the sample injection (after blank subtraction) for each concentration over the commercial purified Protein A (Table 8a and FIG. 8A) and purified recombinant protein A (Table 8b and FIG. 8B). Using this assay format, binding can be assessed to immobilised Protein A (at an immobilisation level of approximately 400RU). A titratable binding response (after blank subtraction) was seen for all constructs carrying human VH3 domains with known positive Protein A binding. Absolute binding responses are dependent on the quality of the immobilised protein A and the level of background signal observed. Titration of a non-binding negative control gives a minimal but measurable binding response up to concentrations of 10 µM.

Non-binding of a test molecule can be confirmed by demonstrating a lack of titratable binding response up to a concentration of 10 µM, with a binding response (at 10 µM) that is no greater than 2-fold higher than the response observed for the negative control at 10 µM.

TABLE 8a

Binding of Fab-dsscFv Molecules to Commercial Purified Secreted Protein A
Binding (RU)

| Concentration (M) | Mu Fab, dsscFv-1 | Mu Fab, dsscFv-2 | Mu Fab, dsscFv-4 | Mu Fab, dsscFv-3B | Mu Fab, dsscFv-3A | Mu Fab, dsscFv-mu1 (Negative Control) |
|---|---|---|---|---|---|---|
| 9.00E-05 | 183.5 | | | | | |
| 3.00E-05 | 128.2 | 8.6 | | 6.4 | 90.4 | 8.5 |
| 1.00E-05 | 79.2 | 2.7 | 326.3 | 2.2 | 51.8 | 2.9 |
| 3.33E-06 | 39.2 | 0.9 | 257.2 | 0.7 | 28.3 | 0.8 |
| 1.11E-06 | 15.8 | 0.3 | 176.8 | 0.3 | 14.1 | 0.3 |
| 3.70E-07 | 5.4 | 0.1 | 100.6 | 0.1 | 6.0 | 0.0 |
| 1.23E-07 | 1.7 | 0.1 | 45.9 | 0.1 | 2.3 | -0.1 |
| 4.12E-08 | 0.4 | 0.1 | 18.0 | 0.2 | 0.9 | 0.0 |
| 1.37E-08 | 0.2 | 0.1 | 6.5 | 0.2 | 0.2 | -0.1 |
| 4.57E-09 | 0.1 | 0.1 | 2.3 | 0.1 | 0.0 | 0.1 |
| 1.52E-09 | | 0.0 | 0.9 | 0.1 | 0.0 | 0.1 |
| 5.10E-10 | | | 0.5 | | | |

TABLE 8b

Binding of Fab-dsscFv Molecules to Purified Recombinant Protein A
Binding (RU)

| Concentration (M) | Mu Fab, dsscFv-1 | Mu Fab, dsscFv-2 | Mu Fab, dsscFv-4 | Mu Fab, dsscFv-3B | Mu Fab, dsscFv-3A | Mu Fab, dsscFv-mu1 (Negative Control) |
|---|---|---|---|---|---|---|
| 9.00E-05 | 726.4 | | | | | |
| 3.00E-05 | 449.7 | 6.8 | | 4.8 | 343.5 | 6.0 |
| 1.00E-05 | 255.3 | 2.3 | 1524.4 | 1.9 | 205.3 | 2.2 |
| 3.33E-06 | 122.8 | 0.8 | 1112.0 | 0.6 | 116.8 | 0.7 |
| 1.11E-06 | 49.2 | 0.2 | 679.2 | 0.2 | 58.3 | 0.3 |
| 3.70E-07 | 17.6 | 0.0 | 343.7 | 0.0 | 24.9 | 0.1 |
| 1.23E-07 | 6.0 | 0.0 | 145.1 | 0.0 | 9.3 | 0.1 |
| 4.12E-08 | 1.9 | 0.0 | 54.6 | 0.0 | 3.2 | 0.1 |
| 1.37E-08 | 0.6 | 0.0 | 19.0 | 0.0 | 1.1 | 0.1 |
| 4.57E-09 | 0.3 | -0.1 | 6.3 | 0.1 | 0.4 | 0.0 |
| 1.52E-09 | | 0.0 | 2.0 | 0.0 | 0.1 | 0.0 |
| 5.10E-10 | | | 0.6 | | | |

1. A multi-specific antibody, comprising:
a polypeptide chain of formula (I): 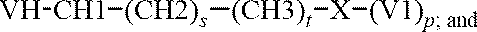
a polypeptide chain of formula (II): 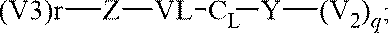
wherein:
VH represents a heavy chain variable domain;
CH1 represents domain 1 of a heavy chain constant region;
CH2 represents domain 2 of a heavy chain constant region;
CH3 represents domain 3 of a heavy chain constant region;
X represents a bond or linker;
V1 represents a dsscFv, a dsFv, a scFv, a VH, a VH or a VHH;
V3 represents a dsscFv, a dsFv, a scFv, a VH, a VH or a VHH;
Z represents a bond or linker;
VL represents a light chain variable domain;
CL represents a domain from a light chain constant region, such as Ckappa;
Y represents a bond or linker;
V2 represents a dsscFv, a dsFv, a scFv, a VH, a VH or a VHH;
p represents 0 or 1 ;
q represents 0 or 1 ;
r represents 0 or 1 ;
s represents 0 or 1 ;
t represents 0 or 1 ;
wherein when p is 0, X is absent and when q is 0, Y is absent and when r is 0, Z is absent; and wherein when q is 0, r is 1 and when r is 0, q is 1 ; and
wherein the polypeptide chain of formula (II) comprises at least one dsscFv, dsFv, scFv, VH or VHH; and
wherein the polypeptide chain of formula (I) comprises a protein A binding domain;
and wherein the polypeptide chain of formula (II) does not bind protein A.

2. A multi-specific antibody according to claim 1, wherein the polypeptide chain of formula (I) comprises one, two or three protein A binding domains.

3. A multi-specific antibody according to claim 1, wherein a protein A binding domain is present in VH and/or CH2-CH3 and/or V1.

4. A multi-specific antibody according to claim 1, wherein the polypeptide chain of formula (I) comprises only one protein A binding domain which is present in VH or V1.

5. A multi-specific antibody according to claim 4, wherein the polypeptide chain of formula (I) comprises only one protein A binding domain which is present in VH.

6. A multi-specific antibody according to claim 4, wherein the polypeptide chain of formula (I) comprises only one protein A binding domain which is present in V1.

7. A multi-specific antibody according to claim 1, wherein the protein A binding domain(s) comprise(s) or consist(s) of a VH3 domain or variant thereof which binds protein A.

8. A multi-specific antibody according to claim 1, wherein V2 and/or V3 do/does not comprise a VH3 domain.

9. A multi-specific antibody according to claim 1, wherein V2 and/or V3, comprise(s) a VH3 domain or variant thereof which does not bind protein A.

10. A multi-specific antibody according to claim 1, wherein p is 1.

11. A multi-specific antibody according to claim 1, wherein q is 1.

12. A multi-specific antibody according to claim 1, wherein r is 1.

13. A multi-specific antibody according to claim 1, wherein q is 0 and r is 1.

14. A multi-specific antibody according claim 1, wherein s is 1, t is 1, p is 0, q is 1, r is 0 and wherein V2 is a dsscFv or dsFv.

15. A multi-specific antibody according to claim 1, wherein s is 0 and t is 0, p is 1, q is 1, r is 0, and wherein V1 and V2 both represent a dsscFv.

16. A multi-specific antibody according to claim 1, wherein V1 binds albumin and comprises a VH3 of sequence SEQ ID NO: 78.

17. A multi-specific antibody according to claim 1, wherein X and/or Y and/or Z is a peptide linker.

18. A multi-specific antibody according to claim 1, wherein V1 and/or V2 and/or V3 are/is a dsscFv or a dsFv, and wherein the light chain and heavy chain variable domains of V1 and/or the light chain and heavy chain variable domains of V2 and/or the light chain and heavy chain variable domains of V3 are linked by a disulfide bond between two engineered cysteine residues, wherein the position of the pair of cysteine residues is selected from the group comprising or consisting of: VH37 and VL95, VH44 and VL100, VH44 and VL105, VH45 and VL87, VH100 and VL50, VH1 00b and VL49, VH98 and VL46, VH101 and VL46, VH1 05 and VL43 and VH106 and VL57 (numbering according to Kabat), wherein the VH and VL values are independently within a given V1 or V2 or V3.

19. A polynucleotide encoding a multi-specific antibody defined in claim 1.

20. A vector comprising a polynucleotide defined in claim 19.

21. A host cell comprising a polynucleotide of claim 19.

22. A host cell comprising at least two vectors, each vector comprising a polynucleotide encoding a different polypeptide chain of a multi-specific antibody defined in claim 1.

23. A pharmaceutical composition comprising a multi-specific antibody according to claim 1 and at least one excipient.

24. (canceled)

25. A method of treating a patient in need thereof, the method comprising administering a therapeutically effective amount of a multi-specific antibody according claim 1.

26. A method of producing a multi-specific antibody comprising a polypeptide chain of formula (I) and a polypeptide chain of formula (II) as defined in claim 1, said method comprising:
a) Expressing a polypeptide chain of formula (I) and a polypeptide chain of formula (II) in a host cell, wherein the polypeptide chain of formula (II) is in excess over the polypeptide chain of formula (I);
b) Recovering the composition of polypeptides expressed at step a), said composition comprising a multi-specific antibody and a LC dimer of formula (II-II); and
c) Purifying the multi-specific antibody, wherein when s is 1 and t is 1, said multi-specific antibody is purified as a dimer with two heavy chains of formula (I) and two associated light chains of formula (II) and, wherein when s is 0 and t is 0, said multi-specific antibody is purified as a dimer with one heavy chain of formula (I) and one associated light chain of formula (II); and,
wherein the polypeptide chain of formula (II) comprises at least one dsscFv, dsFv, scFv, VH or VHH; and,
wherein the polypeptide chain of formula (I) comprises a protein A binding domain; and, wherein the polypeptide chain of formula (II) does not bind protein A; and,
wherein step c) comprises subjecting the composition of polypeptides recovered at step b), optionally following at least one purification step, to a Protein A affinity chromatography column.

27. A method of purifying a multi-specific antibody comprising a polypeptide chain of formula (I) and a polypeptide chain of formula (II) as defined in claim 1, said method comprising:
a) Obtaining a composition of polypeptide chains of formula (I) and polypeptide chains of formula (II) said composition comprising a multi-specific antibody, wherein when s is 1 and t is 1, the multi-specific antibody is a dimer with two heavy chains of formula (I) and two associated light chains of formula (II) and; when s is 0 and t is 0, the multi-specific antibody is a dimer with one heavy chain of formula (I) and one associated light chain of formula (II); and a dimer of two light chains of formula (II-II), associated together (LC dimer); and,
wherein the polypeptide chain of formula (II) comprises at least one dsscFv, dsFv, scFv, VH or VHH; and,
wherein the polypeptide chain of formula (I) comprises a protein A binding domain; and, wherein the polypeptide chain of formula (II) does not bind protein A;
b) Loading the composition obtained in step a), onto a protein A affinity column, such that the multi-specific antibody is retained on the column whilst the LC dimer does not bind to the column;
c) Washing the protein A affinity column;
d) Eluting the multi--specific antibody; and,
e) Recovering the multi-specific antibody.
